(19) United States
(12) Patent Application Publication
(10) Pub. No.: US 2005/0124597 A1
(43) Pub. Date: Jun. 9, 2005

(54) Novel guanidinyl derivatives

(76) Inventors: Rustum Boyce, San Francisco, CA (US); Daniel Chu, Santa Clara, CA (US); David P. Lentini, San Francisco, CA (US)

Correspondence Address: Chiron Corporation;Intellectual Property - R440, P.O. Box 8097, Emeryville, CA, 94662-8097, US

(21) Appl. No.: 10/503,401

(22) PCT Filed: Feb. 3, 2003
(86) PCT No.: PCT/US03/01079

(30) Foreign Application Priority Data
Feb. 4, 2002 (US) 60353183
Jan. 27, 2003 (US) 10351597

(51) Int. Cl. A61K 31/33

(52) U.S. Cl. 514/183

(57) ABSTRACT

A variety of small, guanidino group-containing molecules capable of acting as MC4-R agonists are provided. The compounds have various structures provided herein. The compounds are useful in treating MC4-R mediated diseases and may be formulated into pharmaceutical formulations and compositions.

### FIELD OF THE INVENTION

This invention relates to melanocortin-4 receptor (MC4-R) agonists and methods of their preparation. The invention also relates to methods of treating melanocortin-4 receptor-mediated diseases, such as obesity or diabetes, by activating the melanocortin-4 receptor with compounds provided herein.

### BACKGROUND OF THE INVENTION

Melanocortins are peptide products resulting from post-translational processing of pro-opiomelanocortin and are known to have a broad array of physiological activities. The natural melanocortins include the different types of melanocyte stimulating hormone (α-MSH, β-MSH, γ-MSH) and ACTH. Of these, α-MSH and ACTH are considered to be the main endogenous melanocortins.

The melanocortins mediate their effects through melanocortin receptors (MC-Rs), a subfamily of G-protein coupled receptors. There are at least five different receptor subtypes (MC1-R to MC5-R). MC1-R mediates pigmentation of the hair and skin. MC2-R mediates the effects of ACTH on steroidogenesis in the adrenal gland. MC3-R and MC4-R are predominantly expressed in the brain. MC5-R is considered to have a role in the exocrine gland system.

The melanocortin-4 receptor (MC4-R) is a seven-transmembrane receptor. MC4-R may participate in modulating the flow of visual and sensory information, coordinate aspects of somatomotor control, and/or participate in the modulation of autonomic outflow to the heart. K. G. Mountjoy et al., Science, 257:1248-125 (1992). Significantly, inactivation of this receptor by gene targeting has resulted in mice that develop a maturity onset obesity syndrome associated with hyperphagia, hyperinsulinemia, and hyperglycemia. D. Husznar et al., Cell, 88(1): 131-41 (1997). MC4-R has also been implicated in other disease states including erectile disorders, cardiovascular disorders, neuronal injuries or disorders, inflammation, fever, cognitive disorders, and sexual behavior disorders. M. E. Hadley and C. Haskell-Luevano, The proopiomelanocortin system, Ann. N. Y. Acad. Sci., 885:1 (1999).

Furthermore, observations in connection with endogenous MCx-R antagonists indicate that MC4-R is implicated in endogenous energy regulation. For example, an agouti protein is normally expressed in the skin and is an antagonist of the cutaneous MC receptor involved in pigmentation, MC1-R. M. M. Ollmann et al., Science, 278:135-138 (1997). However, overexpression of agouti protein in mice leads to a yellow coat color due to antagonism of MC1-R and increased food intake and body weight due to antagonism of MC4-R. L. L. Kiefer et al., Biochemistry, 36: 2084-2090 (1997); D. S. Lu et al., Nature, 371:799-802 (1994). Agouti related protein (AGRP), an agouti protein homologue, antagonizes MC4-R but not MC1-R. T. M. Fong et al., Biochem. Biophys. Res. Commun. 237:629-631 (1997). Administration of AGRP in mice increases food intake and causes obesity but does not alter pigmentation. M. Rossi et al., Endocrinology, 139:4428-4431 (1998). Together, this research indicates that MC4-R participates in energy regulation, and therefore, identifies this receptor as a target for a rational drug design for the treatment of obesity.

In connection with MC4-R and its uncovered role in the etiology of obesity and food intake, the prior art includes reports of compounds and compositions that act as agonists or antagonists of MC4-R. As examples, U.S. Pat. No. 6,060,589 describes polypeptides that are capable of modulating signaling activity of melanocortin receptors. Also, U.S. Pat. Nos. 6,054,556 and 5,731,408 describe families of agonists and antagonists for MC4-R receptors that are lactam heptapeptides having a cyclic structure. WO 01/10842 discloses MC4-R binding compounds having a multitude of structures and methods of using such compounds to treat MC4-R associated disorders. Some of the compounds described include amidino- and guanidino-containing arenes and heteroarenes.

Various other classes of compounds have been disclosed as having MC4-R agonist activity. For example, WO 01/70708 and WO 00/74679 disclose MC4-R agonists that are piperidine compounds and derivatives, while WO 01/70337 and WO 99/64002 disclose MC-R agonists that are spiropiperidine derivatives. Other known melanocortin receptor agonists include aromatic amine compounds containing amino acid residues, particularly tryptophan residues, as disclosed in WO 01/55106. Similar agonists are disclosed in WO 01/055107 which comprise aromatic amine compounds containing tertiary amide or tertiary amine groups. Finally, WO 01/055109 discloses melanocortin receptor agonists comprising aromatic amines which are generally bisamides separated by a nitrogen-containing alkyl linker.

Guanidine-containing compounds having a variety of biological activities are also known in the prior art. For example, U.S. Pat. No. 4,732,916 issued to Satoh et al. discloses guanidine compounds useful as antiulcer agents; U.S. Pat. No. 4,874,864, U.S. Pat. No. 4,949,891, and U.S. Pat. No. 4,948,901 issued to Schnur et al. and EP 0343 894 disclose guanidino compounds useful as protease inhibitors and as anti-plasmin and anti-thrombin agents; and U.S. Pat. No. 5,352,704 issued to Okuyama et al. discloses a guanidino compound useful as an antiviral agent. Guanidine-containing compounds are also disclosed in other references. For example, U.S. Pat. No. 6,030,985 issued to Gentile et al discloses guanidine compounds useful for treating and preventing conditions in which inhibition of nitric oxide synthetase is beneficial such as stroke, schizophrenia, anxiety, and pain. U.S. Pat. No. 5,952,381 issued to Chen et al. discloses certain guanidine compounds for use in selectively inhibiting or antagonizing αᵥβ₃ integrins.

Various 5-, 6-, and 7-membered fully saturated 1-azacarbocyclic-2-ylidene derivatives of guanidine are disclosed as having anti-secretory and hypoglycemic activities by U.S. Pat. No. 4,211,867 issued to Rasmussen. Such compounds are also taught as useful for the treatment of cardiovascular disease. Other guanidine derivatives are disclosed by U.S. Pat. No. 5,885,985 issued to Macdonald et al. as useful in therapy to treat inflammation.

Nevertheless, there remains a need for potent and specific agonists of MC4-R that are low molecular weight small molecules. Methods of treating a melanocortin-4 receptor mediated disease, such as obesity, with such small molecules and pharmaceutical formulations containing such small molecules, are also particularly desirable.

### SUMMARY OF THE INVENTION

The instant invention provides potent and specific agonists of MC4-R that are low molecular weight small molecules. Thus, there has been provided, in accordance with various aspects of the invention, compounds of formula IA, IIIA, IIIB, IVA, IVB, VA, VIA, VIIA, VIIB, VIIIA, VIIIB, and IXA as described herein. Also provided are prodrugs of the compounds, pharmaceutically acceptable salts of the compounds, stereoisomers of the compounds, tautomers of the compounds, hydrates of the compounds, hydrides of the compounds, and solvates of the compounds.

One aspect of the invention provides a composition such as a pharmaceutical formulation or medicament that includes at least one of the compounds represented by the above-listed formulas and a pharmaceutically acceptable carrier.

Another aspect of the invention provides a method of treating an MC4-R mediated disease, comprising administering to a subject in need thereof, at least one of the compounds represented by the above-listed formulas. The method may be used to treat diseases which include obesity or type II diabetes.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE PREFERRED EMBODIMENT

The instant invention relates to novel classes of small molecule melanocortin-4 receptor (MC4-R) agonists. These compounds can be formulated into compositions and are useful in activating MC4-R, or in the treatment of MC4-R-mediated diseases, such as obesity, type II diabetes, erectile dysfunction, polycystic ovary disease, complications resulting from or associated with obesity and diabetes, and Syndrome X.

The following definitions are used throughout this specification.

Alkyl groups include straight chain and branched alkyl groups having 1 to about 8 carbon atoms. Examples of straight chain alkyl groups include methyl, ethyl, propyl, butyl, pentyl, hexyl, heptyl, and octyl groups. Examples of branched alkyl groups, include, but not limited to, isopropyl, sec-butyl, t-butyl, and isopentyl groups. Representative substituted alkyl groups may be substituted one or more times with, for example, amino, thio, alkoxy, or halo groups such as F, Cl, Br, and I groups.

Cycloalkyl groups are cyclic alkyl groups such as, but not limited to, cyclopropyl, cyclobutyl, cyclopentyl, cyclohexyl, cycloheptyl, and cyclooctyl groups. Cycloalkyl groups also includes rings that are substituted with straight or branched chain alkyl groups as defined above, and further include cycloalkyl groups that are substituted with other rings including fused rings such as, but not limited to, decalinyl, tetrahydronaphthyl, and indanyl. Cycloalkyl groups also include polycyclic cycloalkyl groups such as, but not limited to, norbornyl, adamantyl, bornyl, camphenyl, isocamphenyl, and carenyl groups. Representative substituted cycloalkyl groups may be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which may be substituted with, for example, alkyl, alkoxy, amino, thio, or halo groups.

Alkenyl groups are straight chain, branched or cyclic lower alkyl groups having 2 to about 8 carbon atoms, and further including at least one double bond, as exemplified, for instance, by vinyl, propenyl, 2-butenyl, 3-butenyl, isobutenyl, cyclohexenyl, cyclopentenyl, cyclohexadienyl, butadienyl, pentadienyl, and hexadienyl groups among others.

Alkynyl groups are straight chain or branched lower alkyl groups having 2 to about 8 carbon atoms, and further including at least one triple bond, as exemplified by groups, including, but not limited to, ethynyl, propynyl, and butynyl groups.

Aryl groups are cyclic aromatic hydrocarbons that do not contain heteroatoms. Thus aryl groups include, but are not limited to, phenyl, azulene, heptalene, biphenylene, indacene, fluorene, phenanthrene, triphenylene, pyrene, naphthacene, chrysene, biphenyl, anthracenyl, and naphthenyl groups. Although the phrase “aryl groups” includes groups containing fused rings, such as fused aromatic-aliphatic ring systems, it does not include aryl groups that have other groups, such as alkyl or halo groups, bonded to one of the ring members. Rather, groups such as tolyl are referred to as substituted aryl groups. The phrase “aryl groups” includes groups bonded to one or more carbon atom(s), and/or nitrogen atom(s), in the compounds of formulas I and II. Representative substituted aryl groups may be mono-substituted or substituted more than once, such as, but not limited to, 2-, 3-, 4-, 5-, or 6-substituted phenyl or benzyl groups, which may be substituted with groups including, but not limited to, amino, alkoxy, alkyl, or halo.

Cycloalkylalkyl groups are alkyl groups as defined above in which a hydrogen or carbon bond of an alkyl group is replaced with a bond to a cycloalkyl group as defined above.

Arylalkyl groups are alkyl groups as defined above in which a hydrogen or carbon bond of an alkyl group is replaced with a bond to an aryl group as defined above.

Heterocyclyl groups are nonaromatic ring compounds containing 3 or more ring members, of which, one or more is a heteroatom such as, but not limited to, N, O, and S. The phrase “heterocyclyl group” includes fused ring species including those comprising fused aromatic and nonaromatic groups. The phrase also includes polycyclic ring systems containing a heteroatom such as, but not limited to quinuclidyl. However, the phrase does not include heterocyclyl groups that have other groups, such as alkyl or halo groups, bonded to one of the ring members. Rather, these are referred to as “substituted heterocyclyl groups”. Heterocyclyl groups include, but are not limited to, piperazino, morpholino, thiomorpholino, pyrrolidino, piperidino and homopiperazino groups. Representative substituted heterocyclyl groups may be mono-substituted or substituted more than once, such as, but not limited to morpholino or piperazino groups, which are 2-, 3-, 4-, 5-, or 6-substituted, or disubstituted with groups including, but not limited to, amino, alkoxy, alkyl, or halo.

Heteroaryl groups are aromatic ring compounds containing 3 or more ring members, of which, one or more is a heteroatom such as, but not limited to, N, O, and S. Heteroaryl groups include, but are not limited to, groups such as furan, thiophene, pyrrole, isopyrrole, diazole, imidazole, isoimidazole, triazole, dithiole, oxathiole, isoxazole, oxazole, thiazole, isothiazole, oxadiazole, oxatriazole, dioxazole, oxathiazole, pyran, dioxin, pyridine, pyrimidine, pyridazine, pyrazine, triazine, oxazine, isoxazine, oxathiazine, azepin, oxepin, thiepin, diazepine, benzofuran, and isobenzofuran. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups including, but not limited to, amino, alkoxy, alkyl, or halo.

Heterocyclylalkyl groups are alkyl groups as defined above in which a hydrogen or carbon bond of an alkyl group is replaced with a bond to a heterocyclyl group as defined above.

Heteroarylalkyl groups are alkyl groups as defined above in which a hydrogen or carbon bond of an alkyl group is replaced with a bond to a heteroaryl group as defined above.

Aminocarbonyl groups are groups of the formula RR′NC(O)—, wherein R or R′ may be the same or different, and each is independently selected from H, or substituted or unsubstituted alkyl, cycloalkyl, aryl, heterocyclyl or heteroaryl groups, as defined above.

In general, “substituted” refers to a group as defined above in which one or more bonds to a hydrogen atom contained therein are replaced by a bond to non-hydrogen or non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups and also substituted cycloalkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles.

Substituted cycloalkyl, substituted aryl, substituted heterocyclyl and substituted heteroaryl also include rings and fused ring systems in which a bond to a hydrogen atom is replaced with a bond to a carbon atom. Therefore, substituted cycloalkyl, substituted aryl, substituted heterocyclyl and substituted heteroaryl groups may be substituted with alkyl groups as defined above.

Pharmaceutically acceptable salts include a salt with an inorganic base, organic base, inorganic acid, organic acid, or basic or acidic amino acid. As salts of inorganic bases, the invention includes, for example, alkali metals such as sodium or potassium, alkali earth metals such as calcium and magnesium or aluminum, and ammonia. As salts of organic bases, the invention includes, for example, trimethylamine, triethylamine, pyridine, picoline, ethanolamine, diethanolamine, triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

Prodrugs, as used in the context of the instant invention, includes those derivatives of the instant compounds which undergo in vivo metabolic biotransformation, by enzymatic or nonenzymatic processes, such as hydrolysis, to form a compound of the invention. Prodrugs can be employed to improve pharmaceutical or biological properties, as for example solubility, melting point, stability and related physicochemical properties, absorption, pharmacodynamics and other delivery-related properties.

The instant invention provides potent and specific agonists of MC4-R that are low molecular weight small molecules. In accordance with one aspect of the invention, the invention provides a first group of compounds of formula IA as shown below.

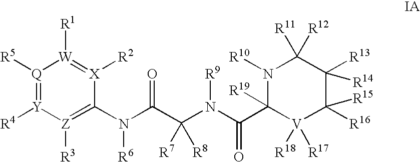

Compounds of the invention further include prodrugs of the first group of compounds of formula IA, pharmaceutically acceptable salts thereof, stereoisomers thereof, tautomers thereof, hydrates thereof, hydrides thereof, and solvates thereof.

In the first group of compounds of formula IA, V is selected from a carbon atom or is absent from the ring such that the carbon atom bonded to R¹⁶ is bonded to the carbon atom bonded to R¹⁹ forming a 5-membered ring.

In the first group of compounds of formula IA, Q, W, X, Y, and Z are independently selected from the group consisting of carbon atoms and nitrogen atoms. In some embodiments of the compounds of formula IA, at least one of Q, W, X, Y, and Z is a nitrogen atom. In other embodiments of the compounds of formula IA, Q, W, X, Y, and Z are all carbon atoms. In other embodiments of the compounds of formula IA, Q is a nitrogen atom and W, X, Y, and Z are all carbon atoms. In other embodiments of the compounds of formula IA, W is a nitrogen atom and Q, X, Y, and Z are all carbon atoms. In other embodiments of the compounds of formula IA, X is a nitrogen atom and Q, W, Y, and Z are all carbon atoms. In still other embodiments of the compounds of formula IA, Y is a nitrogen atom and Q, W, X, and Z are all carbon atoms. In still other embodiments of the compounds of formula IA, Z is a nitrogen atom and Q, W, X, and Y are all carbon atoms.

In the first group of compounds of formula IA, R¹, R², R³, R⁴, and R⁵ may be the same or different, and each may be independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB:

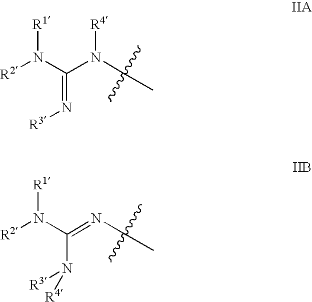

In the first group of compounds of formula IA, R¹ may be absent if W is a nitrogen atom, R² may be absent if X is a nitrogen atom, R³ may be absent if Z is a nitrogen atom, R⁴ may be absent if Y is a nitrogen atom, and R⁵ may be absent if Q is a nitrogen atom. In the first group of compounds of formula IA, at least one of R¹, R², R³, R⁴, or R⁵ is a group having the formula IIA or IIB. In some embodiments of the compounds of formula IA, Q is a carbon atom and R⁵ is a group of formula IIA or IIB.

In the first group of compounds of formula IA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the first group of compounds of formula IA, R^{1′} and R^{2′}, together with the nitrogen to which they are bound, may form a substituted or unsubstituted heterocyclyl or heteroaryl group. In some embodiments of the compounds of formula IA, R^{1′} is H and R^{2′} is selected from the group consisting of substituted and unsubstituted alkyl, arylalkyl, and heteroarylalkyl groups. In other embodiments of the compounds of formula IA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the compounds of formula IA, R^{1′} and R^{2′} may be the same or different and are each independently selected from the group consisting of substituted and unsubstituted alkyl, arylalkyl, and heteroarylalkyl groups. In various other embodiments of the compounds of formula IA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In yet other embodiments of the compounds of formula IA, R^{1′} and R^{2′}, together with the nitrogen to which they are bound, form a substituted or unsubstituted heterocyclyl group. In still other embodiments of the compounds of formula IA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In another embodiment of the compounds of formula IA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still another embodiment of the compounds of formula IA, R^{1′} and R^{2′}, together with the nitrogen to which they are bound, form a substituted or unsubstituted heterocyclyl ring containing at least one oxygen heteroatom. In still other embodiments of the compounds of formula IA, R^{1′} and R^{2′}, together with the nitrogen to which they are bound, form a substituted or unsubstituted piperazino, morpholino, pyrrolidino, piperidino, homopiperazino, or azepino group. In still further embodiments of the compounds of formula IA, R^{1′} and R^{2′}, together with the nitrogen to which they are bound, form a substituted piperazino group optionally substituted by one or two alkyl groups, for example, one or two methyl groups.

In the first group of compounds of formula IA, R^{3′} is selected from the group consisting of H, and substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In some embodiments of the first group of compounds of formula IA, R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In one embodiment of the compounds of formula IA, R^{3′} is selected from the group consisting of substituted and unsubstituted cycloalkyl, polycyclic cycloalkyl, alkenyl, alkyl, and aryl groups. In still other embodiments of the compounds of formula IA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the compounds of formula IA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethylnorbornyl, 4-isopropylcyclohexyl, and 3-methylcycloheptyl groups.

In the first group of compounds of formula IA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the compounds of formula IA, R^{4′} is H.

In the first group of compounds of formula IA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the compounds of formula IA, R⁶ is H.

In the first group of compounds of formula IA, R⁷ is selected from the group consisting of H, substituted and unsubstituted alkyl groups, substituted and unsubstituted cycloalkyl groups, and substituted and unsubstituted aryl groups. In various embodiments of the compounds of formula IA, R⁷ is H.

In the first group of compounds of formula IA, R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups. In one embodiment of the compounds of formula IA, R⁸ is selected from the group consisting of substituted and unsubstituted arylalkyl groups, and substituted and unsubstituted heteroarylalkyl groups. In some embodiments of compounds of formula IA, R⁸ is a substituted or unsubstituted phenylalkyl group, a substituted or unsubstituted pyridylalkyl group, or a substituted or unsubstituted indolylalkyl group. In other embodiments of the compounds of formula IA, R⁸ is a substituted or unsubstituted phenylalkyl group or a substituted or unsubstituted indolylalkyl group. In these embodiments of the compounds of formula IA, R⁸ may be a 2,4-disubstituted phenylmethyl group or an indolylmethyl group. In certain embodiments where R⁸ is a substituted arylalkyl or heteroarylalkyl, such as a phenylalkyl or pyridylalkyl, one substituent on the aryl or heteroaryl ring is a group having the formula IIA or IIB, wherein R^{1′}, R^{2′}, R^{3′}, and R^{4′} have the characteristics described above. In some embodiments of the compounds of formula IA, R⁸ is selected from the group consisting of 2,4-dihalophenylmethyl, and 2,4-dialkylphenylmethyl groups. In still other embodiments of the compounds of formula IA, R⁸ is selected from the group consisting of phenylmethyl, 2,4-dichlorophenylmethyl, 4-methoxyphenylmethyl, 4-bromophenylmethyl, 4-methylphenylmethyl, 4-chlorophenylmethyl, 4-ethylphenylmethyl, cyclohexenylmethyl, 2-methoxyphenylmethyl, 2-chlorophenylmethyl, 2-fluorophenylmethyl, 3-methoxyphenylmethyl, 3-fluorophenylmethyl, thienylmethyl, indolylmethyl, 4-hydroxyphenylmethyl, 3,4-dimethoxyphenylmethyl, 2-chloro-4-iodophenylmethyl, 2-fluoro4-methylphenylmethyl, 2-fluoro-4-bromophenylmethyl, 2-fluoro-4-methoxyphenylmethyl, 2-trifluoromethyl-4-fluorophenylmethyl, 2,4-difluorophenylmethyl, 2,4-dimethylphenylmethyl, and 2,4-dimethoxyphenylmethyl groups.

In the first group of compounds of formula IA, R⁹ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the compounds of formula IA, R⁹ is H.

In the first group of compounds of formula IA, R¹⁰ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the compounds of formula IA, R¹⁰ is H.

In the first group of compounds of formula IA, R¹¹, R¹², R¹³, R¹⁶, R¹⁷, and R¹⁸ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups, substituted and unsubstituted amide groups, substituted and unsubstituted sulfone groups, and substituted and unsubstituted sulfonamide groups. In the first group of compounds of formula IA, R¹³ and R¹⁶ together can represent a double bond between the carbon atoms bonded to R¹³ and R⁶.

In the first group of compounds of formula IA, R¹⁴ and R¹⁵ are selected from the group consisting of H, and substituted and unsubstituted alkyl groups. In the first group of compounds of formula IA, R¹⁴ and R¹⁵ together with the two carbon atoms to which they are bound form a substituted or unsubstituted, saturated or unsaturated, carbocyclic or heterocyclic ring comprising 5, 6, or 7 members. In one embodiment of the compounds of formula IA, R¹⁴ and R¹⁵, together with the two carbon atoms to which they are bound, form a substituted or unsubstituted carbocyclic ring comprising 6 members.

In the first group of compounds of formula IA where V is absent, R¹⁷ and R¹⁸ are also absent.

In the first group of compounds of formula IA, R¹⁹ is selected from the group consisting of H, substituted and unsubstituted alkyl groups, substituted and unsubstituted cycloalkyl groups, and substituted and unsubstituted aryl groups. In various embodiments of the compounds of formula IA, R¹⁹ is H.

In accordance with another aspect of the invention, the invention provides a second group of compounds of formula IIIA, as shown below.

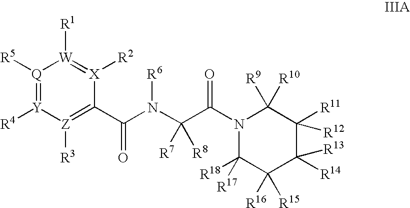

Compounds of the invention further include prodrugs of the second group of compounds of formula IIIA, pharmaceutically acceptable salts thereof, stereoisomers thereof, tautomers thereof, hydrates thereof, hydrides thereof, and solvates thereof.

In the second group of compounds of formula IIIA, Q, W, X, Y, and Z are independently selected from the group consisting of carbon atoms and nitrogen atoms. In some embodiments of the compounds of formula IIIA, at least one of Q, W, X, Y, and Z is a nitrogen atom. In other embodiments of the compounds of formula IIIA, Q, W, X, Y, and Z are all carbon atoms. In other embodiments of the compounds of formula IIIA, Q is a nitrogen atom and W, X, Y, and Z are all carbon atoms. In other embodiments of the compounds of formula IIIA, W is a nitrogen atom and Q, X, Y, and Z are all carbon atoms. In other embodiments of the compounds of formula IIIA, X is a nitrogen atom and Q, W, Y, and Z are all carbon atoms. In still other embodiments of the compounds of formula IIIA, Y is a nitrogen atom and Q, W, X, and Z are all carbon atoms. In still other embodiments of the compounds of formula IIIA, Z is a nitrogen atom and Q, W, X, and Y are all carbon atoms.

In the second group of compounds of formula IIIA, R¹, R², R³, R⁴, and R⁵ may be the same or different, and each may be independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

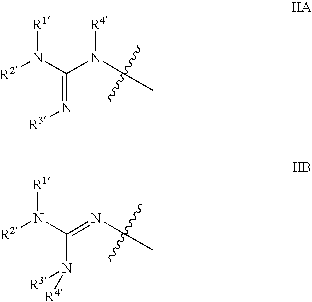

In the second group of compounds of formula IIIA, R¹ may be absent if W is a nitrogen atom, R² may be absent if X is a nitrogen atom, R³ may be absent if Z is a nitrogen atom, R⁴ may be absent if Y is a nitrogen atom, and R⁵ may be absent if Q is a nitrogen atom. In the second group of compounds of formula IIIA, one of R¹, R², R³, R⁴, or R⁵ is a group having the formula IIA or IIB. In some embodiments of the compounds of formula IIIA, Q is a carbon atom and R⁵ is a group of formula IIA or IIB.

In the second group of compounds of formula IIIA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is be selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the second group of compounds having the formula IIA, R^{1′} and R^{2′}, together with the nitrogen to which they are bound, may alternatively form a substituted or unsubstituted heterocyclyl or heteroaryl group. In some embodiments of the second group of compounds of formula IIIA, R^{1′} is H and R^{2′} is selected from the group consisting of substituted and unsubstituted alkyl, arylalkyl, and heteroarylalkyl groups. In other embodiments of the second group of compounds of formula IIIA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the second group of compounds of formula IIIA, R^{1′} and R^{2′} may be the same or different and are each independently selected from the group consisting of substituted and unsubstituted alkyl, arylalkyl, and heteroarylalkyl groups. In various other embodiments of the second group of compounds of formula IIIA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the second group of compounds of formula IIIA, R^{1′} and R^{2′}, together with the nitrogen to which they are bound, form a substituted or unsubstituted heterocyclyl group. In still other embodiments of the second group of compounds of formula IIIA, R^{1′} and R^{2′}, together with the nitrogen to which they are bound form a substituted or unsubstituted saturated heterocyclyl group comprising at least one heteroatom selected from the group consisting of O, S, and N, in addition to the nitrogen atom to which R^{1′} and R^{2′} are bound. In still other embodiments of the second group of compounds of formula IIIA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments of the second group of compounds of formula IIIA, R^{1′} and R^{2′}, together with the nitrogen to which they are bound, form a substituted or unsubstituted heterocyclyl ring containing at least one oxygen heteroatom. Representative examples of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. This includes compounds wherein R^{1′} and R^{2′}, together with the nitrogen to which they are bound, form a substituted piperazino group optionally substituted by one or two alkyl groups, for example, one or two methyl groups.

In the second group of compounds of formula IIIA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In various embodiments of the second group of compounds of formula IIIA, R^{3′} is selected from the group consisting of substituted and unsubstituted cycloalkyl, polycyclic cycloalkyl, alkenyl, alkyl, and aryl groups. In other embodiments of the second group of compounds of formula IIIA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the second group of compounds of formula IIIA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethyinorbornyl, 4-isopropylcyclohexyl, and 3-methylcycloheptyl groups.

In the second group of compounds of formula IIIA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the second group of compounds of formula IIIA, R^{4′} is H.

In the second group of compounds of formula IIIA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the second group of compounds of formula IIIA, R⁶ is H.

In the second group of compounds of formula IIIA, R⁷ is selected from the group consisting of H, substituted and unsubstituted alkyl groups, substituted and unsubstituted cycloalkyl groups, and substituted and unsubstituted aryl groups. In various embodiments of the second group of compounds of formula IIIA, R⁷ is H.

In the second group of compounds of formula IIIA, R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups. In some embodiments of the second group of compounds of formula IIIA, R⁸ is selected from the group consisting of substituted and unsubstituted arylalkyl groups, and substituted and unsubstituted heteroarylalkyl groups. In some embodiments of compounds of formula IIIA, R⁸ is a substituted or unsubstituted phenylalkyl group, a substituted or unsubstituted pyridylalkyl group, or a substituted or unsubstituted indolylalkyl group. In other embodiments of the second group of compounds of formula IIIA, R⁸ is a substituted or unsubstituted phenylalkyl group or a substituted or unsubstituted indolylalkyl group. More specifically, R⁸ may be a 2,4-disubstituted phenylmethyl group or an indolylmethyl group. In some embodiments of the second group of compounds of formula IIIA, R⁸ is selected from the group consisting of 2,4-dihalophenylmethyl, and 2,4-dialkylphenylmethyl groups. In certain embodiments where R⁸ is a substituted arylalkyl or heteroarylalkyl, such as a phenylalkyl or pyridylalkyl, one substituent on the aryl or heteroaryl ring is a group having the formula IIA or IIB, wherein R^{1′}, R^{2′}, R^{3′}, and R^{4′} have the characteristics described above. In certain other embodiments of the second group of compounds of formula IIIA, R⁸ is selected from the group consisting of phenylmethyl, 2,4-dichlorophenylmethyl, 4-methoxyphenylmethyl, 4-bromophenylmethyl, 4-methylphenylmethyl, 4-chlorophenylmethyl, 4-ethylphenylmethyl, cyclohexenylmethyl, 2-methoxyphenylmethyl, 2-chlorophenylmethyl, 2-fluorophenylmethyl, 3-methoxyphenylmethyl, 3-fluorophenylmethyl, thienylmethyl, indolylmethyl, 4-hydroxyphenylmethyl, 3,4-dimethoxyphenylmethyl, 2-chloro-4-iodophenylmethyl, 2-fluoro4-methylphenylmethyl, 2-fluoro4-bromophenylmethyl, 2-fluoro-4-methoxyphenylmethyl, 2-trifluoromethyl-4-fluorophenylmethyl, 2,4-difluorophenylmethyl, 2,4-dimethylphenylmethyl, or 2,4-dimethoxyphenylmethyl groups.

In the second group of compounds of formula IIIA, R⁹, R¹⁰, R¹¹, R¹², R¹⁵, R¹⁶, R¹⁷ and R¹⁸ are selected from the group consisting of H, Cl, F, Br, I, —CN, —OH, —NO₂, substituted and unsubstituted alkoxy groups, and substituted and unsubstituted alkyl groups.

In the second group of compounds of formula IIIA, R¹³ and R¹⁴ are selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups, and substituted and unsubstituted alkylsulfonylalkyl groups. In compounds of the second group having the formula IIIA, R¹³ and R¹⁴ may alternatively join together with the carbon to which they are bound to form a substituted or unsubstituted carbocyclic or heterocyclic ring.

In some embodiments of the second group of compounds of formula IIIA, the compound of formula IIIA has the formula IIIB, as shown below. 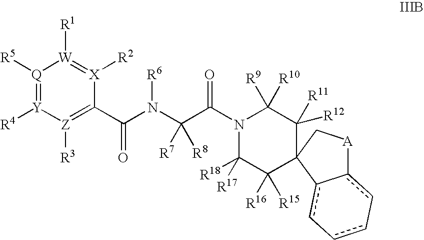
wherein the dotted lines in the 6-membered carbocyclic ring indicate that the 6-membered ring is a substituted or unsubstituted cyclohexyl or benzene ring In this embodiment, A may be independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups. In these embodiments, the variables in the formula IIIB have the same definition as defined above with respect to compounds of formula IIIA.

In various embodiments of compounds of formula IIIB wherein the 6-membered carbocyclic ring is a substituted benzene ring, one substituent on the benzene ring is a group having the formula IIA or IIB, wherein R^{1′}, R^{2′}, R^{3′}, and R^{4′} have the characteristics described above.

In accordance with yet another aspect of the invention, the invention provides a third group of compounds of formula IVA, as shown below.

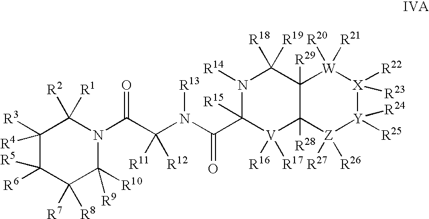

Compounds of the invention further include prodrugs of the third group of compounds of formula IVA, pharmaceutically acceptable salts thereof, stereoisomers thereof, tautomers thereof, hydrates thereof, hydrides thereof, and solvates thereof.

In the third group of compounds of formula IVA, V is selected from a carbon atom or is absent from the ring such that the carbon atom bonded to R²⁸ is bonded to the carbon atom bonded to R¹⁵ forming a 5-membered ring.

In the third group of compounds of formula IVA, W, X, Y, and Z are independently selected from the group consisting of carbon atoms and nitrogen atoms. In some embodiments of the third group of compounds of formula IVA, at least one of W, X, Y, and Z is a nitrogen atom whereas in other embodiments W, X, Y, and Z are all carbon atoms.

In the third group of compounds of formula IVA, R¹, R², R³, R⁴, R⁷, R⁸, R⁹, and R¹⁰ are independently selected from the group consisting of H, Cl, F, Br, I, OH, —CN, —NO₂ substituted and unsubstituted alkoxy groups, and substituted and unsubstituted alkyl groups.

In the third group of compounds of formula IVA, R⁵ and R⁶ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups, and substituted and unsubstituted alkylsulfonylalkyl groups. In other compounds of the third group of compounds of formula IVA, R⁵ and R⁵ may alternatively join together with the carbon atom to which they are both bound to form a substituted or unsubstituted carbocyclic or heterocyclic ring.

In the third group of compounds of formula IVA, R¹¹ is selected from the group consisting of H, substituted and unsubstituted alkyl groups, substituted and unsubstituted cycloalkyl groups, and substituted and unsubstituted aryl groups. In various embodiments of the third group of compounds of formula IVA, R¹¹ is H.

In the third group of compounds of formula IVA, R¹² is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups. In one embodiment of the third group of compounds of formula IVA, R¹² is selected from the group consisting of substituted and unsubstituted arylalkyl groups, and substituted and unsubstituted heteroarylalkyl groups. In some embodiments of compounds of formula IVA, R¹² is a substituted or unsubstituted phenylalkyl group, a substituted or unsubstituted pyridylalkyl group, or a substituted or unsubstituted indolylalkyl group. In other embodiments of the third group of compounds of formula IVA, R¹² is a substituted or unsubstituted phenylalkyl group or a substituted or unsubstituted indolylalkyl group. Such embodiments include those in which R¹² is a 2,4-disubstituted phenylmethyl group or an indolylmethyl group. These embodiments of the third group of compounds of formula IVA include those in which R¹² is selected from the group consisting of 2,4-dihalophenylmethyl, and 2,4-dialkylphenylmethyl groups. In certain embodiments where R¹² is a substituted arylalkyl or heteroarylalkyl, such as a phenylalkyl or pyridylalkyl, one substituent on the aryl or heteroaryl ring is a group having the formula IIA or IIB, wherein R^{1′}, R^{2′}, R^{3′}, and R^{4′} have the characteristics described above. In still other embodiments of the third group of compounds of formula IVA, R¹² is selected from the group consisting of phenylmethyl, 2,4-dichlorophenylmethyl, 4-methoxyphenylmethyl, 4-bromophenylmethyl, 4-methylphenylmethyl, 4-chlorophenylmethyl, 4-ethylphenylmethyl, cyclohexenylmethyl, 2-methoxyphenylmethyl, 2-chlorophenylmethyl, 2-fluorophenylmethyl, 3-methoxyphenylmethyl, 3-fluorophenylmethyl, thienylmethyl, indolylmethyl, 4-hydroxyphenylmethyl, 3,4-dimethoxyphenylmethyl, 2-chloro-4-iodophenylmethyl, 2-fluoro4-methylphenylmethyl, 2-fluoro-4-bromophenylmethyl, 2-fluoro-4-methoxyphenylmethyl, 2-trifluoromethyl4-fluorophenylmethyl, 2,4-difluorophenylmethyl, 2,4-dimethylphenylmethyl, or 2,4-dimethoxyphenylmethyl groups.

In the third group of compounds of formula IVA, R¹³ and R¹⁴ are independently selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the third group of compounds of formula IVA, R¹³ and/or R¹⁴ is(are) H.

In the third group of compounds of formula IVA, R¹⁵, R^{16,} R¹⁷, R¹⁸, and R¹⁹ are independently selected from H, substituted and unsubstituted alkyl groups, substituted and unsubstituted cycloalkyl groups, and substituted and unsubstituted aryl groups. In the third group of compounds of formula IVA where V is absent, R¹⁵ and R¹⁷ are also absent.

In the third group of compounds of formula IVA, R²⁰, R²², R²⁴, and, R²⁶ are independently selected from the group consisting of H, substituted and unsubstituted alkyl groups, and groups having the formula IIA or IIB:

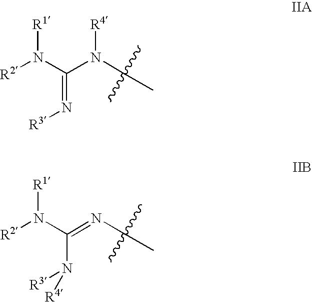

In the third group of compounds of formula IVA, R²⁰ may be absent if W is a nitrogen atom, R²² may be absent if X is a nitrogen atom, R²⁶ may be absent if Z is a nitrogen atom, and R²⁴ may be absent if Y is a nitrogen atom. In the third group of compounds of formula IVA, at least one of R²⁰, R²², R²⁴ or R²⁶ is a group having the formula IA or IIB. In some such embodiments, one of R²⁰, R²², R²⁴ or R²⁶ is a group having the formula IIA or IIB.

In the third group of compounds having the formula IVA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} may be selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the third group of compounds of formula IVA, R^{1′} and R^{2′}, together with the nitrogen to which they are bound, may alternatively form a substituted or unsubstituted heterocyclyl or heteroaryl group. In some embodiments of the third group of compounds of formula IVA, R^{1′} is H and R^{2′} is selected from the group consisting of substituted and unsubstituted alkyl, arylalkyl, and heteroarylalkyl groups. In other embodiments of the third group of compounds of formula IVA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the third group of compounds of formula IVA, R^{1′} and R^{2′} may be the same or different and are each independently selected from the group consisting of substituted and unsubstituted alkyl, arylalkyl, and heteroarylalkyl groups. In various other embodiments of the third group of compounds of formula IVA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the third group of compounds of formula IVA, R^{1′} and R^{2′}, together with the nitrogen to which they are bound form a substituted or unsubstituted heterocyclyl group. In still other embodiments of the third group of compounds of formula IVA, R^{1′} and R^{2′}, together with the nitrogen to which they are bound form a substituted or unsubstituted saturated heterocyclyl group comprising at least one heteroatom selected from the group consisting of O, S, and N, in addition to the nitrogen atom to which R^{1′} and R^{2′} are bound. In other embodiments of the third group of compounds of formula IVA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments of the third group of compounds of formula IVA, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. Other embodiments of the third group of compounds of formula IVA include those in which R^{1′} and R^{2′}, together with the nitrogen to which they are bound, form a substituted piperazino group optionally substituted by one or two alkyl groups, for example, one or two methyl groups.

In the third group of compounds having the formula IVA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In some embodiments of the third group of compounds of formula IVA, R^{3′} is selected from the group consisting of substituted and unsubstituted cycloalkyl, polycyclic cycloalkyl, alkenyl, alkyl, and aryl groups. In other embodiments of the third group of compounds of formula IVA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the third group of compounds of formula IVA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethylnorbornyl, 4-isopropylcyclohexyl, and 3-methylcycloheptyl groups.

In the third group of compounds having the formulas IVA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the third group of compounds of formula IVA, R^{4′} is H. In some embodiments of the third group of compounds, one, some or all of R⁴′, R¹¹, R¹³, R¹⁴, and R¹⁵ is (are) all H.

In the third group of compounds of formula IVA, R²¹, R²³, R²⁵, and R²⁷ are independently selected from the group consisting of H, substituted and unsubstituted alkyl groups, substituted and unsubstituted cycloalkyl groups, and substituted and unsubstituted aryl groups. In the third group of compounds having the formula IVA, R²¹ and R²³ together can alternatively represent a double bond between the carbons bonded to R²¹ and R²³. In compounds of the third group having the formula IVA, R²⁵ and R²⁷ together can alternatively represent a double bond between the carbons bonded to R²⁵ and R²⁷.

In the third group of compounds of formula IVA, R²⁸ and R²⁹ are independently selected from the group consisting of H, and substituted and unsubstituted alkyl groups. In the third group of compounds of formula IVA, R²⁸ and R²⁹ together can alternatively represent a double bond between the carbon atoms bonded to R²⁸ and R²⁹.

In one embodiment of the third group of compounds of formula IVA, the compound of formula IVA has the formula IVB below 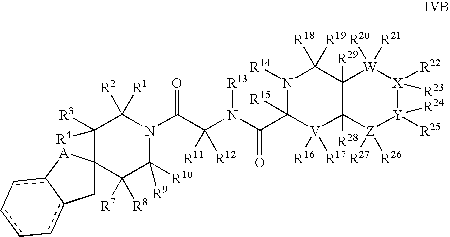
wherein the dotted lines in the 6-membered carbocyclic ring indicate that the 6-membered ring is a substituted or unsubstituted cyclohexyl or benzene ring. In these embodiments, A may be independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups. In these embodiments the variables in the formula IVB have the same definition as defined above with respect to compounds of formula IVA.

In various embodiments of compounds of formula IVB wherein the 6-membered carbocyclic ring is a substituted benzene ring, one substituent on the benzene ring is a group having the formula IIA or IIB, wherein R^{1′}, R^{2′}, R^{3′}, and R^{4′} have the characteristics described above.

In accordance with yet another aspect of the invention, the invention provides a fourth group of compounds of formula VA, as shown below.

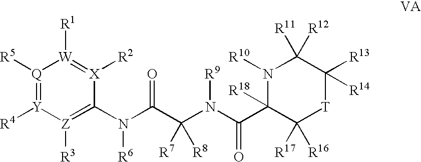

Compounds of the invention further include prodrugs of the fourth group of compounds of formula VA, pharmaceutically acceptable salts thereof, stereoisomers thereof, tautomers thereof, hydrates thereof, hydrides thereof, and solvates thereof.

In the fourth group of compounds of formula VA, T is selected from the group consisting of O, S, and NR¹⁵ groups;

In the fourth group of compounds of formula VA, Q, W, X, Y, and Z are independently selected from the group consisting of carbon atoms and nitrogen atoms. In some embodiments of the fourth group of compounds of formula VA, at least one of Q, W, X, Y, and Z is a nitrogen atom. In other embodiments of the fourth group of compounds of formula VA, Q, W, X, Y, and Z are all carbon atoms. In other embodiments of the fourth group of compounds of formula VA, Q is a nitrogen atom and W, X, Y, and Z are all carbon atoms. In other embodiments of the fourth group of compounds of formula VA, W is a nitrogen atom and Q, X, Y, and Z are all carbon atoms. In other embodiments of the fourth group of compounds of formula VA, X is a nitrogen atom and Q, W, Y, and Z are all carbon atoms. In still other embodiments of the fourth group of compounds of formula VA, Y is a nitrogen atom and Q, W, X, and Z are all carbon atoms. In still other embodiments of the fourth group of compounds of formula VA, Z is a nitrogen atom and Q, W, X, and Y are all carbon atoms.

In the fourth group of compounds of formula VA, R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB:

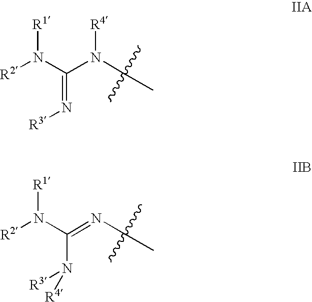

In the fourth group of compounds of formula VA, R¹ may be absent if W is a nitrogen atom, R² may be absent if X is a nitrogen atom, R³ may be absent if Z is a nitrogen atom, R⁴ may be absent if Y is a nitrogen atom, and R⁵ may be absent if Q is a nitrogen atom. In the fourth group of compounds of formula VA, at least one of R¹, R², R³, R⁴, or R⁵ is a group having the formula IIA or IIB. In one embodiment of the fourth group of compounds of formula VA, Q is a carbon atom and R⁵ is a group of formula IIA or IIB. In some embodiments of the fourth group of compounds of formula VA, one of R¹, R², R³, R⁴, or R⁵ is a group having the formula IIA or IIB.

In the fourth group of compounds of formula VA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the fourth group of compounds of formula VA, R^{1′} and R^{2′}, together with the nitrogen to which they are bound, may alternatively form a substituted or unsubstituted heterocyclyl or heteroaryl group. In some embodiments of the fourth group of compounds of formula VA, R^{1′} is H and R^{2′} is selected from the group consisting of substituted and unsubstituted alkyl, arylalkyl, and heteroarylalkyl groups. In other embodiments of the fourth group of compounds of formula VA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the fourth group of compounds of formula VA, R^{1′} and R^{2′} may be the same or different and are each independently selected from the group consisting of substituted and unsubstituted alkyl, arylalkyl, and heteroarylalkyl groups. In various other embodiments of the fourth group of compounds of formula VA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In one embodiment of the fourth group of compounds of formula VA, R^{1′} and R^{2′}, together with the nitrogen to which they are bound, form a substituted or unsubstituted heterocyclyl group. In still other embodiments of the fourth group of compounds of formula IVA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In other embodiments of the fourth group of compounds of formula VA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments of the fourth group of compounds of formula VA, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. Other embodiments of the fourth group of compounds of formula VA include those in which R^{1′} and R^{2′}, together with the nitrogen to which they are bound, form a substituted piperazino group optionally substituted by one or two alkyl groups, for example, one or two methyl groups.

In the fourth group of compounds of formula VA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In some embodiments of the fourth group of compounds of formula VA, R^{3′} is selected from the group consisting of substituted and unsubstituted cycloalkyl, polycyclic cycloalkyl, alkenyl, alkyl, and aryl groups. This embodiment contemplates that R^{3′} may be selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In other embodiments of the fourth group of compounds of formula VA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethyinorbornyl, 4-isopropylcyclohexyl, and 3-methylcycloheptyl groups.

In the fourth group of compounds of formula VA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the fourth group of compounds of formula VA, R^{4′} is H.

In the fourth group of compounds of formula VA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments, of the fourth group of compounds of formula VA R⁶ is H.

In the fourth group of compounds of formula VA, R⁷ is selected from the group consisting of H, substituted and unsubstituted alkyl groups, substituted and unsubstituted cycloalkyl groups, and substituted and unsubstituted aryl groups. In various embodiments of the fourth group of compounds of formula VA, R⁷ is H.

In the fourth group of compounds of formula VA, R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups. In one embodiment of the fourth group of compounds of formula VA, R⁸ is selected from the group consisting of substituted and unsubstituted arylalkyl groups, and substituted and unsubstituted heteroarylalkyl groups. In some embodiments of compounds of formula VA, R⁸ is a substituted or unsubstituted phenylalkyl group, a substituted or unsubstituted pyridylalkyl group, or a substituted or unsubstituted indolylalkyl group. In other embodiments of the fourth group of compounds of formula VA, R⁸ is a substituted or unsubstituted phenylalkyl group or a substituted or unsubstituted indolylalkyl group. In these embodiments of the fourth group of compounds of formula VA, R⁸ may be a 2,4-disubstituted phenylmethyl group or an indolylmethyl group. These embodiments contemplate that R³ may be selected from the group consisting of 2,4-dihalophenylmethyl, and 2,4-dialkylphenylmethyl groups. In certain embodiments where R⁸ is a substituted arylalkyl or heteroarylalkyl, such as a phenylalkyl or pyridylalkyl, one substituent on the aryl or heteroaryl ring is a group having the formula IIA or IIB, wherein R^{1′}, R^{2′}, R^{3′}, and R^{4′} have the characteristics described above. In other embodiments of the fourth group of compounds of formula VA, R⁸ is selected from the group consisting of phenylmethyl, 2,4-dichlorophenylmethyl, 4-methoxyphenylmethyl, 4-bromophenylmethyl, 4-methylphenylmethyl, 4-chlorophenylmethyl, 4-ethylphenylmethyl, cyclohexenylmethyl, 2-methoxyphenylmethyl, 2-chlorophenylmethyl, 2-fluorophenylmethyl, 3-methoxyphenylmethyl, 3-fluorophenylmethyl, thienylmethyl, indolylmethyl, 4-hydroxyphenylmethyl, 3,4-dimethoxyphenylmethyl, 2-chloro-4-iodophenylmethyl, 2-fluoro-4-methylphenylmethyl, 2-fluoro-4-bromophenylmethyl, 2-fluoro-4-methoxyphenylmethyl, 2-trifluoromethyl-4-fluorophenylmethyl, 2,4-difluorophenylmethyl, 2,4-dimethylphenylmethyl, or 2,4-dimethoxyphenylmethyl groups.

In the fourth group of compounds of formula VA, R⁹ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the fourth group of compounds of formula VA, R⁹ is H.

In the fourth group of compounds of formula VA, R¹⁰ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the fourth group of compounds of formula VA, R¹⁰ is H.

In the fourth group of compounds of formula VA, R¹¹, R¹², R¹³, R¹⁴, R¹⁶, and R¹⁷ are selected from the group consisting of H, Cl, F, Br, I, —CN, —OH, —NO₂, substituted and unsubstituted aryl groups, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups, substituted and unsubstituted amide groups, substituted and unsubstituted sulfone groups, and substituted and unsubstituted sulfonamide groups.

In the fourth group of compounds of formula VA, R¹⁵ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups.

In one embodiment, T is an NR¹⁵ group and R¹⁴ and R¹⁵ together with the atoms to which they are bound form a substituted or unsubstituted heterocyclic ring comprising 6 members.

In the fourth group of compounds of formula VA, R¹² and R¹⁴ together with the carbon atoms to which they are bound, may form a substituted or unsubstituted, saturated or unsaturated carbocyclic or heterocyclic ring comprising 5 or 6 members. In the fourth group of compounds of formula VA, R¹⁴ and R¹⁵ together with the atoms to which they are bound, may form a substituted or unsubstituted, saturated or unsaturated heterocyclic ring comprising 5 or 6 members.

In the fourth group of compounds of formula VA, R¹⁸ is selected from the group consisting of H, substituted and unsubstituted alkyl groups, substituted and unsubstituted cycloalkyl groups, and substituted and unsubstituted aryl groups. In various embodiments of the fourth group of compounds of formula VA, R¹⁸ is H.

In accordance with another aspect of the invention, the invention provides a fifth group of compounds of formula VIA as shown below.

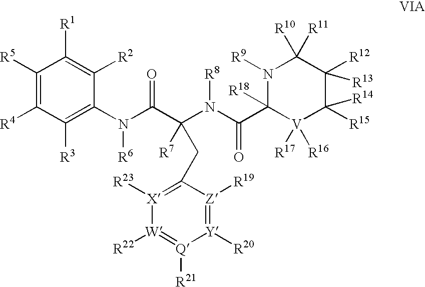

Compounds of the invention further include prodrugs of the fifth group of compounds of formula VIA, pharmaceutically acceptable salts thereof, stereoisomers thereof, tautomers thereof, hydrates thereof, hydrides thereof, and solvates thereof.

In the fifth group of compounds of formula VIA, V is selected from a carbon atom or is absent from the ring such that the carbon atom bonded to R¹⁵ is bonded to the carbon atom bonded to R¹⁸ forming a 5-membered ring.

In the fifth group of compounds of formula VIA, Q′, W′, X′, Y′, and Z′ are independently selected from the group consisting of carbon atoms and nitrogen atoms. In some embodiments of the fifth group of compounds of formula VIA, at least one of Q′, W′, X′, Y′, and Z′ is a nitrogen atom. In other embodiments of the fifth group of compounds of formula VIA, Q′, W′, X′, Y′, and Z′ are all carbon atoms. In other embodiments of the fifth group of compounds of formula VIA, Q′ is a nitrogen atom and W′, X′, Y′, and Z′ are all carbon atoms. In other embodiments of the fifth group of compounds of formula VIA, W′ is a nitrogen atom and Q′, X′, Y′, and Z′ are all carbon atoms. In other embodiments of the fifth group of compounds of formula VIA, X′ is a nitrogen atom and Q′, W′, Y′, and Z′ are all carbon atoms. In still other embodiments of the fifth group of compounds of formula VIA, Y′ is a nitrogen atom and Q′, W′, X′, and Z′ are all carbon atoms. In still other embodiments of the fifth group of compounds of formula VIA, Z′ is a nitrogen atom and Q′, W′, X′, and Y′ are all carbon atoms.

In the fifth group-of compounds of formula VIA, R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

In the fifth group of compounds of formula VIA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the fifth group of compounds of formula VIA, R⁶ is H.

In the fifth group of compounds of formula VIA, R⁷ is selected from the group consisting of H, substituted and unsubstituted alkyl groups, substituted and unsubstituted cycloalkyl groups, and substituted and unsubstituted aryl groups. In various embodiments of the fifth group of compounds of formula VIA, R⁷ is H.

In the fifth group of compounds of formula VIA, R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups.

In the fifth group of compounds of formula VIA, R⁹ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the fifth group of compounds of formula VIA, R⁹ is H.

In the fifth group of compounds of formula VIA, R¹⁰, R¹¹, R¹², R¹⁵, R¹⁶, and R¹⁷ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups, substituted and unsubstituted amide groups, substituted and unsubstituted sulfone groups, and substituted and unsubstituted sulfonamide groups. In the fifth group of compounds of formula VIA, R¹² and R¹⁵ together may alternatively represent a double bond between the carbon atoms bonded to R¹² and R¹⁵.

In the fifth group of compounds of formula VIA, R¹³ and R¹⁴ are selected from the group consisting of H, and substituted and unsubstituted alkyl groups. Alternatively, R¹³ and R¹⁴ together with the two carbon atoms to which they are bound form a substituted or unsubstituted, saturated or unsaturated, carbocyclic or heterocyclic ring comprising 5, 6, or 7 members. In one embodiment of the fifth group of compounds of formula VIA, R¹³ and R¹⁴, together with the two carbon atoms to which they are bound, form a substituted or unsubstituted carbocyclic ring comprising 6 members.

In the fifth group of compounds of formula VIA, R¹⁸ is selected from the group consisting of H, substituted and unsubstituted alkyl groups, substituted and unsubstituted cycloalkyl groups, and substituted and unsubstituted aryl groups. In various embodiments of the fifth group of compounds of formula VIA, R¹⁸ is H.

In compounds of formula VIA where V is absent, R¹⁶ and R¹⁷ are also absent.

In the fifth group of compounds of formula VIA, R¹⁹, R²⁰, R²¹, R²², and R²³ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups and groups of formula IIA or IIB.

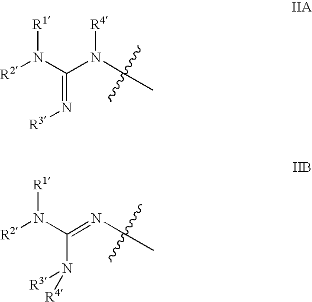

In the fifth group of compounds of formula VIA, R²² may be absent if W′ is a nitrogen atom, R²³ may be absent if X′ is a nitrogen atom, R¹⁹ may be absent if Z′ is a nitrogen atom, R²⁰ may be absent if Y′ is a nitrogen atom, and R²¹ may be absent if Q′ is a nitrogen atom. In the fifth group of compounds of formula VIA, at least one of R¹⁹, R²⁰, R²¹, R²² or R²³ is a group having the formula IIA or IIB. In some embodiments of the fifth group of compounds of formula VIA, Q′ is a carbon atom and R²¹ is a group of formula IIA or IIB. In some embodiments of the fifth group of compounds of formula VIA, one of R¹⁹, R²⁰, R²¹, R²², or R²³ is a group having the formula IIA or IIB

In the fifth group of compounds having the formula VIA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the fifth group of compounds having the formula VIA, R^{1′} and R^{2′}, together with the nitrogen to which they are bound, may alternatively form a substituted or unsubstituted heterocyclyl or heteroaryl group. In some embodiments of the fifth group of compounds of formula VIA, R^{1′} is H and R^{2′} is selected from the group consisting of substituted and unsubstituted alkyl, arylalkyl, and heteroarylalkyl groups. In other embodiments of the fifth group of compounds of formula VIA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the fifth group of compounds of formula VIA, R^{1′} and R^{2′} may be the same or different and are each independently selected from the group consisting of substituted and unsubstituted alkyl, arylalkyl, and heteroarylalkyl groups. In various other embodiments of the fifth group of compounds of formula VIA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the fifth group of compounds of formula VIA, R^{1′} and R^{2′}, together with the nitrogen to which they are bound, form a substituted or unsubstituted heterocyclyl group. In some embodiments of the fifth group of compounds of formula VIA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In other embodiments of the fifth group of compounds of formula VIA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments of the fifth group of compounds of formula VIA, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. Other embodiments of the fifth group of compounds of formula VIA include those in which R^{1′} and R^{2′}, together with the nitrogen to which they are bound, form a substituted piperazino group optionally substituted by one or two alkyl groups, for example, one or two methyl groups.

In the fifth group of compounds having the formula VIA, R^{3′} is selected from the group consisting of H, and substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In some embodiments of the fifth group of compounds having the formula VIA, R^{3′} is selected from the group consisting of substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groupsIn some embodiments of the fifth group of compounds of formula VIA, R^{3′} is selected from the group consisting of substituted and unsubstituted cycloalkyl, polycyclic cycloalkyl, alkenyl, alkyl, and aryl groups. In other embodiments of the fifth group of compounds of formula VIA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the fifth group of compounds of formula VIA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethylnorbornyl, 4-isopropylcyclohexyl, and 3-methylcycloheptyl groups.

In the fifth group of compounds of formula VIA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the fifth group of compounds of formula VIA, R^{4′} is H.

In accordance with another aspect of the invention, the invention provides a sixth group of compounds of formula VIIA, as shown below.

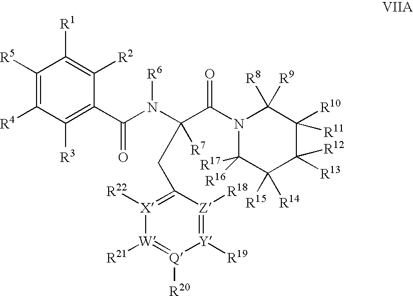

Compounds of the invention further include prodrugs of the sixth group of compounds of formula VIIA, pharmaceutically acceptable salts thereof, stereoisomers thereof, tautomers thereof, hydrates thereof, hydrides thereof, and solvates thereof.

In the sixth group of compounds of formula VIIA, Q′, W′, X′, Y′, and Z′ are independently selected from the group consisting of carbon atoms and nitrogen atoms. In some embodiments of the sixth group of compounds of formula VIIA, at least one of Q′, W′, X′, Y′, and Z′ is a nitrogen atom. In other embodiments of the sixth group of compounds of formula VIIA, Q′, W′, X′, Y′, and Z′ are all carbon atoms. In other embodiments of the sixth group of compounds of formula VIIA, Q′ is a nitrogen atom and W′, X′, Y′, and Z′ are all carbon atoms. In other embodiments of the sixth group of compounds of formula VIIA, W′ is a nitrogen atom and Q′, X′, Y′, and Z′ are all carbon atoms. In other embodiments of the sixth group of compounds of formula VIIA, X′ is a nitrogen atom and Q′, W′, Y′, and Z′ are all carbon atoms. In still other embodiments of the sixth group of compounds of formula VIIA, Y′ is a nitrogen atom and Q′, W′, X′, and Z′ are all carbon atoms. In still other embodiments of the sixth group of compounds of formula VIIA, Z′ is a nitrogen atom and Q′, W′, X′, and Y′ are all carbon atoms.

In the sixth group of compounds of formula VIIA, R¹, R², R³, R⁴, and R⁵ may be the same or different, and are each independently selected from the group consisting of H. Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

In the sixth group of compounds of formula VIIA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the sixth group of compounds of formula VIIA, R⁶ is H.

In the sixth group of compounds of formula VIIA, R⁷ is selected from the group consisting of H, substituted and unsubstituted alkyl groups, substituted and unsubstituted cycloalkyl groups, and substituted and unsubstituted aryl groups. In various embodiments of the sixth group of compounds of formula VIIA, R⁷ is H.

In the sixth group of compounds of formula VIIA, R⁸, R⁹, R¹⁰, R¹¹, R¹⁴, R¹⁵, R¹⁶ and R¹⁷ are selected from the group consisting of H, Cl, F, Br, I, —CN, —OH, —NO₂, substituted and unsubstituted alkoxy groups, and substituted and unsubstituted alkyl groups.

In the sixth group of compounds of formula VIIA, R¹² and R¹³ are selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups, and substituted and unsubstituted alkylsulfonylalkyl groups. In the sixth group of compounds of formula VIIA, R¹² and R¹³ may alternatively join together with the carbon to which they are bound to form a substituted or unsubstituted carbocyclic or heterocyclic ring including carbocyclic or heterocyclic rings substituted with a group of formula IA or IIB.

In the sixth group of compounds of formula VIIA, R¹⁸, R¹⁹, R²⁰, R²¹, and R²² may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

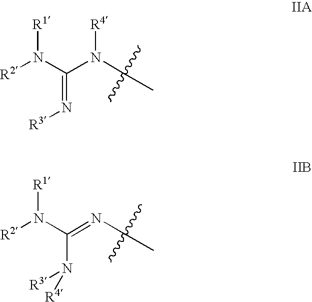

In the sixth group of compounds of formula VIIA, R²¹ may be absent if W′ is a nitrogen atom, R²² may be absent if X′ is a nitrogen atom, R¹⁸ may be absent if Z′ is a nitrogen atom, R¹⁹ may be absent if Y′ is a nitrogen atom, and R²⁰ may be absent if Q′ is a nitrogen atom. In the sixth group of compounds of formula VIIA, at least one of R¹⁸, R¹⁹, R²⁰, R²¹, or R²² is a group having the formula IIA or IIB. In one embodiment of the sixth group of compounds of formula VIIA, Q′ is a carbon atom and R²⁰ is a group of formula IIA or IIB. In some embodiments of the sixth group of compounds of formula VIIA, one of R¹⁸, R¹⁹, R²⁰, R²¹, or R²² is a group having the formula IIA or IIB

In the sixth group of compounds of formula VIIA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the sixth group of compounds having the formula VIIA, R^{1′} and R^{2′}, together with the nitrogen to which they are bound, may alternatively form a substituted or unsubstituted heterocyclyl or heteroaryl group. In one embodiment of the sixth group of compounds of formula VIIA, R^{1′} is H and R^{2′} is selected from the group consisting of substituted and unsubstituted alkyl, arylalkyl, and heteroarylalkyl groups. In other embodiments of the sixth group of compounds of formula VIIA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the sixth group of compounds of formula VIIA, R^{1′} and R^{2′} may be the same or different and are each independently selected from the group consisting of substituted and unsubstituted alkyl, arylalkyl, and heteroarylalkyl groups. In various embodiments of the sixth group of compounds of formula VIIA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In other embodiments of the sixth group of compounds of formula VIIA, R^{1′} and R^{2′}, together with the nitrogen to which they are bound form a substituted or unsubstituted heterocyclyl group. In still other embodiments of the sixth group of compounds of formula VIIA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In other embodiments of the sixth group of compounds of formula VIIA, R^{1′} and R^{2′}, together with the nitrogen to which they are bound, form a substituted or unsubstituted heterocyclyl ring containing at least one nitrogen heteroatom in addition to the nitrogen atom to which R^{1′} and R²′ are both bound. In still other embodiments of the sixth group of compounds of formula VIIA, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. In other embodiments of the sixth group of compounds of formula VIIA, R^{1′} and R^{2′}, together with the nitrogen to which they are bound, form a substituted piperazino group optionally substituted by one or two alkyl groups, for example, one or two methyl groups.

In the sixth group of compounds having the formula VIIA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In some embodiments of the sixth group of compounds of formula VIIA, R^{3′} is selected from the group consisting of substituted and unsubstituted cycloalkyl, polycyclic cycloalkyl, alkenyl, alkyl, and aryl groups. In other embodiments of the sixth group of compounds of formula VIIA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the sixth group of compounds of formula VIIA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethylnorbornyl, 4-isopropylcyclohexyl, and 3-methylcycloheptyl groups.

In the sixth group of compounds having the formula VIIA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the sixth group of compounds of formula VIIA, R^{4′} is H.

In one embodiment of the sixth group of compounds of formula VIIA, the compound of formula VIIA has the formula VIIB, as shown below. 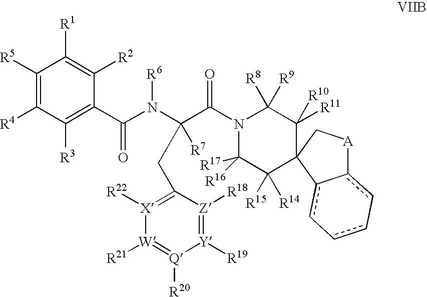
wherein the dotted lines in the 6-membered carbocyclic ring indicate that the 6-membered ring is a substituted or unsubstituted cyclohexyl or benzene ring. In this embodiment, A may be independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups. In these embodiments the variables in the formula VIIB have the same definition as defined above with respect to compounds of formula IVA.

In various embodiments of compounds of formula VIIB wherein the 6-membered carbocyclic ring is a substituted benzene ring, one substituent on the benzene ring is a group having the formula IIA or IIB, wherein R^{1′}, R^{2′}, R^{3′}, and R^{4′} have the characteristics described above.

In accordance with yet -another aspect of the invention, the invention provides a seventh group of compounds of formula VIIIA, as shown below.

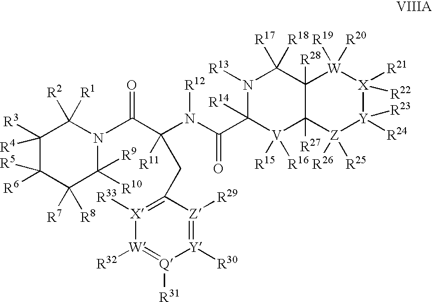

Compounds of the invention further include prodrugs of the seventh group of compounds having the formula VIIIA, pharmaceutically acceptable salts thereof, stereoisomers thereof, tautomers thereof, hydrates thereof, hydrides thereof, and solvates thereof.

In the seventh group of compounds of formula VIIIA, V is selected from a carbon atom or is absent from the ring such that the carbon atom bonded to R²⁷ is bonded to the carbon atom bonded to R¹⁴ forming a 5-membered ring.

In the seventh group of compounds of formula VIIIA, W, X, Y, and Z are independently selected from the group consisting of carbon atoms and nitrogen atoms.

In the seventh group of compounds of formula VIIIA, Q′, W′, X′, Y′, and Z′ are independently selected from the group consisting of carbon atoms and nitrogen atoms. In some embodiments of the seventh group of compounds of formula VIIIA, at least one of Q′, W′, X′, Y′, and Z′ is a nitrogen atom. In other embodiments of the seventh group of compounds of formula VIIIA, Q′, W′, X′, Y′, and Z′ are all carbon atoms. In other embodiments of the seventh group of compounds of formula VIIIA, Q′ is a nitrogen atom and W′, X′, Y′, and Z′ are all carbon atoms. In other embodiments of the seventh group of compounds of formula VIIIA, W′ is a nitrogen atom and Q′, X′, Y′, and Z′ are all carbon atoms. In other embodiments of the seventh group of compounds of formula VIIIA, X′ is a nitrogen atom and Q′, W′, Y′, and Z′ are all carbon atoms. In still other embodiments of the seventh group of compounds of formula VIIIA, Y′ is a nitrogen atom and Q′, W′, X′, and Z′ are all carbon atoms. In still other embodiments of the seventh group of compounds of formula VIIIA, Z′ is a nitrogen atom and Q′, W′, X′, and Y′ are all carbon atoms.

In the seventh group of compounds of formula VIIIA, R¹, R², R³, R⁴, R⁷, R⁸, R⁹, and R¹⁰ are independently selected from the group consisting of H, Cl, F, Br, I, OH, —CN, —NO₂ substituted and unsubstituted alkoxy groups, and substituted and unsubstituted alkyl groups.

In the seventh group of compounds of formula VIIIA, R⁵ and R⁶ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups, and substituted and unsubstituted alkylsulfonylalkyl groups. In the seventh group of compounds of formula VIIIA, R⁵ and R⁶ may alternatively join together with the carbon to which they are bound form a substituted or unsubstituted carbocyclic or heterocyclic ring including carbocyclic or heterocyclic rings substituted with a group of formula IIA or IIB.

In the seventh group of compounds of formula VIIIA, R¹¹ is selected from the group consisting of H, substituted and unsubstituted alkyl groups, substituted and unsubstituted cycloalkyl groups, and substituted and unsubstituted aryl groups. In various embodiments of the seventh group of compounds of formula VIIIA, R¹¹ is H.

In the seventh group of compounds of formula VIIIA, R¹² and R¹³ are independently selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the seventh group of compounds of formula VIIIA, R¹² and/or R¹³ is(are) H.

In the seventh group of compounds of formula VIIIA, R¹⁴, R¹⁵, R¹⁶, R¹⁷, and R¹⁸ are independently selected from H, substituted and unsubstituted alkyl groups, substituted and unsubstituted cycloalkyl groups, and substituted and unsubstituted aryl groups. In the seventh group of compounds of formula VIIIA where V is absent, R¹⁵ and R¹⁶ are also absent.

In the seventh group of compounds of formula VIIIA, R¹⁹, R²⁰, R²¹, R²², R²³, R²⁴, R²⁵, and, R²⁶ are independently selected from the group consisting of H, substituted and unsubstituted alkyl groups, substituted and unsubstituted cycloalkyl groups, and substituted and unsubstituted aryl groups. In the seventh group of compounds of formula VIIIA, R¹⁹ may be absent if W is a nitrogen atom, R²¹ may be absent if X is a nitrogen atom, R²⁵ may be absent if Z is a nitrogen atom, and R²³ may be absent if Y is a nitrogen atom. In the seventh group of compounds of formula VIIIA, R²⁰ and R²² together can alternatively represent a double bond between the carbons bonded to R²⁰ and R²². In the seventh group of compounds of formula VIIIA, R²⁴ and R²⁶ together can alternatively represent a double bond between the carbons bonded to R²⁴ and R²⁶.

In the seventh group of compounds of formula VIIIA, R²⁷ and R²⁸ are independently selected from the group consisting of H and substituted and unsubstituted alkyl groups. In the seventh group of compounds of formula VIIIA, R²⁷ and R²⁸ together may alternatively represent a double bond between the carbon atoms bonded to R²⁷ and R²⁸.

In the seventh group of compounds of formula VIIIA, R²⁹, R³⁰, R³¹, R³² and, R³³ may be the same or different and are independently selected from the group consisting of H, Cl, I, F, Br, OH, NH₂° CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups having the formula IIA or IIB.

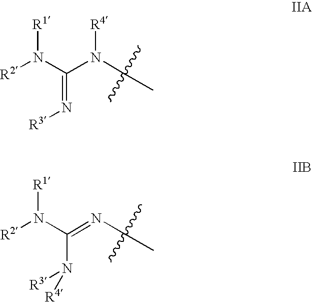

In the seventh group of compounds of formula VIIIA, R³¹ may be absent if Q′ is a nitrogen atom, R³² may be absent if W′ is a nitrogen atom, R³³ may be absent if X′ is a nitrogen atom, R²⁹ may be absent if Z′ is a nitrogen atom, and R³⁰ may be absent if Y′ is a nitrogen atom. In the seventh group of compounds of formula VIIIA, at least one of R²⁹, R³⁰, R³¹, R³² or R³³ is a group having the formula IIA or IIB. In some embodiments of the seventh group of compounds of formula VIIIA, one of R²⁹, R³⁰, R³¹, R³², or R³³ is a group having the formula IIA or IIB.

In the seventh group of compounds having the formula VIIIA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the seventh group of compounds of formula VIIIA, R^{1′} and R^{2′}, together with the nitrogen to which they are bound, may alternatively form a substituted or unsubstituted heterocyclyl or heteroaryl group. In one embodiment of the seventh group of compounds of formula VIIIA, R^{1′} is H and R^{2′} is selected from the group consisting of substituted and unsubstituted alkyl, arylalkyl, and heteroarylalkyl groups. In other embodiments of the seventh group of compounds of formula VIIIA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the seventh group of compounds of formula VIIIA, R^{1′} and R^{2′} may be the same or different and are each independently selected from the group consisting of substituted and unsubstituted alkyl, arylalkyl, and heteroarylalkyl groups. In various other embodiments of the seventh group of compounds of formula VIIIA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the seventh group of compounds of formula VIIIA, R^{1′} and R^{2′}, together with the nitrogen to which they are bound, form a substituted or unsubstituted heterocyclyl group. In still further embodiments of the seventh group of compounds of formula VIIIA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In other embodiments of the seventh group of compounds of formula VIIIA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments of the seventh group of compounds of formula VIIIA, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. Still other embodiments of the seventh group of compounds having the formula VIIIA include those in which R^{1′} and R^{2′}, together with the nitrogen to which they are bound, form a substituted piperazino group optionally substituted by one or two alkyl groups, for example, one or two methyl groups.

In the seventh group of compounds having the formula VIIIA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In various embodiments of the seventh group of compounds of formula VIIIA, R^{3′} is selected from the group consisting of substituted and unsubstituted cycloalkyl, polycyclic cycloalkyl, alkenyl, alkyl, and aryl groups. In other embodiments of the seventh group of compounds of formula VIIIA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In still other embodiments of the seventh group of compounds of formula VIIIA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethyinorbornyl, 4-isopropylcyclohexyl, and 3-methylcycloheptyl groups.

In the seventh group of compounds having the formulas VIIIA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the seventh group of compounds of formula VIIIA, R^{4′} is H.

In one embodiment of the seventh group of compounds of formula VIIIA, the compound of formula VIIIA has the formula VIIIB below 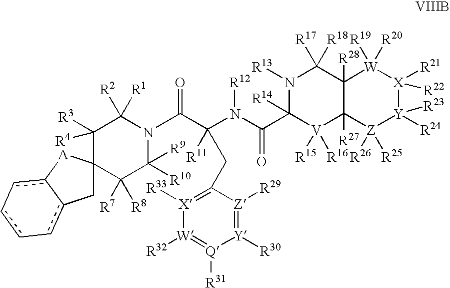
wherein the dotted lines in the 6-membered carbocyclic ring indicate that the 6-membered ring is a substituted or unsubstituted cyclohexyl or benzene ring. In these embodiments, A may be independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups. In these embodiments the variables in the formula VIIIB have the same definition as defined above with respect for compounds of formula VIIIA.

In various embodiments of compounds of formula VIIIB wherein the 6-membered carbocyclic ring is a substituted benzene ring, one substituent on the benzene ring is a group having the formula IIA or IIB, wherein R^{1′}, R^{2′}, R^{3′}, and R^{4′} have the characteristics described above

In accordance with yet another aspect of the invention, the invention provides an eighth group of compounds of formula IXA, as shown below.

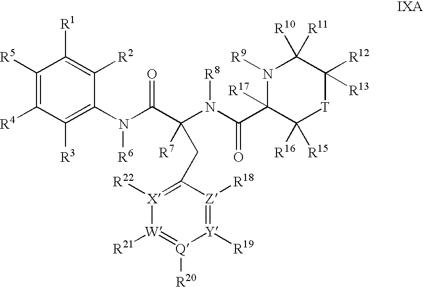

Compounds of the invention further include prodrugs of the eighth group of compounds of formula IXA, pharmaceutically acceptable salts thereof, stereoisomers thereof, tautomers thereof, hydrates thereof, hydrides thereof, and solvates thereof.

In the eighth group of compounds of formula IXA, T may be selected from the group consisting of O, S, and NR¹⁴ groups.

In the eighth group of compounds of formula IXA, Q′, W′, X′, Y′, and Z′ are independently selected from the group consisting of carbon atoms and nitrogen atoms. In some embodiments of the eighth group of compounds of formula IXA, at least one of Q′, W′, X′, Y′, and Z′ is a nitrogen atom. In other embodiments of the eighth group of compounds of formula IXA, Q′, W′, X′, Y′, and Z′ are all carbon atoms. In other embodiments of the eighth group of compounds of formula IXA, Q′ is a nitrogen atom and W′, X′, Y′, and Z′ are all carbon atoms. In other embodiments of the eighth group of compounds of formula IXA, W′ is a nitrogen atom and Q′, X′, Y′, and Z′ are all carbon atoms. In other embodiments of the eighth group of compounds of formula IXA, X′ is a nitrogen atom and Q′, W′, Y′, and Z′ are all carbon atoms. In still other embodiments of the eighth group of compounds of formula IXA, Y′ is a nitrogen atom and Q′, W′, X′, and Z′ are all carbon atoms. In still other embodiments of the eighth group of compounds of formula IXA, Z′ is a nitrogen atom and Q′, W′, X′, and Y′ are all carbon atoms.

In the eighth group of compounds of formula IXA, R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

In the eighth group of compounds of formula IXA, R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the eighth group of compounds of formula IXA, R⁶ is H.

In the eighth group of compounds of formula IXA, R⁷ is selected from the group consisting of H, substituted and unsubstituted alkyl groups, substituted and unsubstituted cycloalkyl groups, and substituted and unsubstituted aryl groups. In various embodiments of the eighth group of compounds of formula IXA, R⁷ is H.

In the eighth group of compounds of formula IXA, R⁸ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the eighth group of compounds of formula IXA, R⁸ is H.

In the eighth group of compounds of formula IXA, R⁹ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups. In various embodiments of the eighth group of compounds of formula IXA, R⁹ is H.

In the eighth group of compounds of formula IXA, R¹⁰, R¹¹, R¹², R¹³, R¹⁵, and R¹⁶ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups, substituted and unsubstituted amide groups, substituted and unsubstituted sulfone groups, and substituted and unsubstituted sulfonamide groups.

In the eighth group of compounds of formula IXA, R¹⁴ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups.

In one embodiment of the eighth group of compounds of formula IXA, T is an NR¹⁴ group and R¹³ and R¹⁴ together with the two atoms to which they are bound form a substituted or unsubstituted heteroocyclic ring comprising 6 members.

In the eighth group of compounds of formula IXA, R¹¹ and R¹³ together with the carbon atoms to which they are bound, may alternatively form a substituted or unsubstituted, saturated or unsaturated carbocyclic or heterocyclic ring comprising 5 or 6 members. In the eighth group of compounds of formula IXA, R¹³ and R¹⁴ together with the atoms to which they are bound, may alternatively form a substituted or unsubstituted, saturated or unsaturated heterocyclic ring comprising 5 or 6 members;

In the eighth group of compounds of formula IXA, R¹⁷ is selected from the group consisting of H, substituted and unsubstituted alkyl groups, substituted and unsubstituted cycloalkyl groups, and substituted and unsubstituted aryl groups. In various embodiments of the eighth group of compounds of formula IXA, R¹⁷ is H.

In the eighth group of compounds of formula IXA, R¹⁸, R¹⁹, R²⁰, R²¹, and R²² may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB.

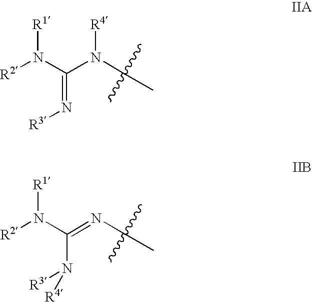

In the eighth group of compounds of formula IXA, R²¹ may be absent if W′ is a nitrogen atom, R²² may be absent if X′ is a nitrogen atom, R¹⁸ may be absent if Z′ is a nitrogen atom, R¹⁹ may be absent if Y′ is a nitrogen atom, and R²⁰ may be absent if Q′ is a nitrogen atom. In the eighth group of compounds of formula IXA, at least one of R¹⁸, R¹⁹, R²⁰, R²¹, or R²² is a group having the formula IIA or IIB. In one embodiment of the eighth group of compounds of formula IXA, Q′ is a carbon atom and R²⁰ is a group of formula IIA or IIB. In some embodiments of the eighth group of compounds of formula IXA, at one of R¹⁸, R¹⁹, R²⁰, R²¹ or R²² is a group having the formula IIA or IIB

In the eighth group of compounds of formula IXA, R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups, and R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups. In the eighth group of compounds of formula IXA, R^{1′} and R^{2′}, together with the nitrogen to which they are bound, form a substituted or unsubstituted heterocyclyl or heteroaryl group. In some embodiments of the eighth group of compounds of formula IXA, R^{1′} is H and R^{2′} is selected from the group consisting of substituted and unsubstituted alkyl, arylalkyl, and heteroarylalkyl groups. In other embodiments of the eighth group of compounds of formula IXA, R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In still other embodiments of the eighth group of compounds of formula IXA, R^{1′} and R^{2′} may be the same or different and are each independently selected from the group consisting of substituted and unsubstituted alkyl, arylalkyl, and heteroarylalkyl groups. In various embodiments of the eighth group of compounds of formula IXA, R^{1′} and R^{2′} may be the same or different and are each independently selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups. In one embodiment of the eighth group of compounds of formula IXA, R^{1′} and R^{2′}, together with the nitrogen to which they are bound, form a substituted or unsubstituted heterocyclyl group. In other embodiments of the eighth group of compounds of formula VIIIA, R^{1′} and R^{2′}, together with the nitrogen to which they are bound, form a substituted or unsubstituted saturated heterocyclyl group comprising at least one heteroatom selected from the group consisting of O, S, and N, in addition to the nitrogen atom to which R^{1′} and R^{2′} are bound. In other embodiments of the eighth group of compounds of formula IXA, R^{1′} and R^{2′}, together with the nitrogen to which they are bound, form a substituted or unsubstituted heterocyclyl ring containing at least one nitrogen heteroatom in addition to the nitrogen atom to which R^{1′} and R^{2′} are both bound. In still other embodiments, R^{1′} and R^{2′}, together with the nitrogen to which they are bound, form a substituted or unsubstituted heterocyclyl ring containing at least one oxygen heteroatom. Representative examples of some of the above-described heterocyclyl embodiments include those for which R^{1′} and R^{2′}, together with the nitrogen to which they are bound, form a substituted or unsubstituted piperazino, morpholino, pyrrolidino, piperidino, homopiperazino, or azepino group. Other embodiments of the eighth group of compounds of formula IXA include those in which R^{1′} and R^{2′}, together with the nitrogen to which they are bound, form a substituted piperazino group optionally substituted by one or two alkyl groups, for example, one or two methyl groups.

In the eighth group of compounds of formula IXA, R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups. In one embodiment of the eighth group of compounds of formula IXA, R^{3′} is selected from the group consisting of substituted and unsubstituted cycloalkyl, polycyclic cycloalkyl, alkenyl, alkyl, and aryl groups. In other embodiments of the eighth group of compounds of formula IXA, R^{3′} is selected from the group consisting of substituted and unsubstituted cyclohexyl, 2-alkylcyclohexyl, 2,2-dialkylcyclohexyl, 2,3-dialkylcyclohexyl, 2,4-dialkylcyclohexyl, 2,5-dialkylcyclohexyl, 2,6-dialkylcyclohexyl, 3,4-dialkylcyclohexyl, 3-alkylcyclohexyl, 4-alkylcyclohexyl, 3,3,5-trialkylcyclohexyl, cyclohexylmethyl, 2-aminocyclohexyl, 3-aminocyclohexyl, 4-aminocyclohexyl, 2,3-diaminocyclohexyl, 2,4-diaminocyclohexyl, 3,4-diaminocyclohexyl, 2,5-diaminocyclohexyl, 2,6-diaminocyclohexyl, 2,2-diaminocyclohexyl, 2-alkoxycyclohexyl, 3-alkoxycyclohexyl, 4-alkoxycyclohexyl, 2,3-dialkoxycyclohexyl, 2,4-dialkoxycyclohexyl, 3,4-dialkoxycyclohexyl, 2,5-dialkoxycyclohexyl, 2,6-dialkoxycyclohexyl, 2,2-dialkoxycyclohexyl, 2-alkylthiocyclohexyl, 3-alkylthiocyclohexyl, 4-alkylthiocyclohexyl, 2,3-dialkylthiocyclohexyl, 2,4-dialkylthiocyclohexyl, 3,4-dialkylthiocyclohexyl, 2,5-dialkylthiocyclohexyl, 2,6-dialkylthiocyclohexyl, 2,2-dialkylthiocyclohexyl, cyclopentyl, cycloheptyl, cyclohexenyl, isopropyl, n-butyl, cyclooctyl, 2-arylcyclohexyl, 2-phenylcyclohexyl, 2-arylalkylcyclohexyl, 2-benzylcyclohexyl, 4-phenylcyclohexyl, adamantyl, isocamphenyl, carenyl, 7,7-dialkylnorbornyl, bornyl, norbornyl, and decalinyl groups. In other embodiments of the eighth group of compounds of formula IXA, R^{3′} is selected from the group consisting of substituted and unsubstituted cyclohexyl, 2-methylcyclohexyl, 2,2-dimethylcyclohexyl, 2,3-dimethylcyclohexyl, 2,4-dimethylcyclohexyl, 2,5-dimethylcyclohexyl, 2,6-dimethylcyclohexyl, 3,4-dimethylcyclohexyl, 3-methylcyclohexyl, 4-methylcyclohexyl, cyclohexenyl, 3,3,5-trimethylcyclohexyl, 4-t-butylcyclohexyl, 2-methylcycloheptyl, cyclohexylmethyl, isopinocampheyl, 7,7-dimethylnorbornyl, 4-isopropylcyclohexyl, and 3-methylcycloheptyl groups.

In the eighth group of compounds of formula IXA, R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups. In various embodiments of the eighth group of compounds of formula IXA, R^{4′} is H.

Another aspect of the invention provides a composition comprising at least one of the compounds represented by the above-listed formulas and a pharmaceutically acceptable carrier.

Another aspect of the invention provides a method of treating an MC4-R mediated disease, comprising administering to a subject in need thereof, at least one of the compounds represented by the above-listed formulas. The method may be used to treat diseases and complications arising from diseases which include obesity or type II diabetes. Additionally, the method may be used to treat erectile dysfunction, polycystic ovary disease, and Syndrome X.

The compounds of the invention may generally be assembled through peptide couplings. The reagents and conditions employed in these couplings to form amide bonds are familiar to one of skill in the art. Examples of these coupling conditions can also be found in the review article “Chemical Synthesis of Natural Product Peptides: Coupling Methods for the Incorporation of Noncoded Amino Acids into Peptides” (Humphrey, J. M.; Chamberlin, A. R.; Chem. Rev. 1997, 97, p 2243-2256).

One aspect of the invention involves coupling three building blocks: an amino subunit, a central amino acid moiety, and an acyl subunit. The amino subunit is first coupled with the central amino acid moiety containing a nitrogen protecting group. Various common nitrogen protecting groups, such as those listed in “Protective Groups in Organic Synthesis 2^{nd} ed.” (Greene, T. W.; Wuts, P. G. M; John Wiley & Sons, Inc.; New York: 1991), may be useful in increasing the efficiency of the coupling reaction with respect to solubility, yield, and chemical and optical purity. After removing the protecting group following coupling, the exposed nitrogen is then coupled with the acyl subunit. Additional functional group or protecting group manipulations may also be required either before or after the coupling steps to generate certain compounds of the invention. Further examples of starting materials for the amino, central amino acid, and acyl subunits and their preparations may be found in WO 00/74679, WO 99/64002, WO 01/70337, and WO 01/70708 which are hereby incorporated by reference. The above-described process can be summarized in the following synthesis scheme:

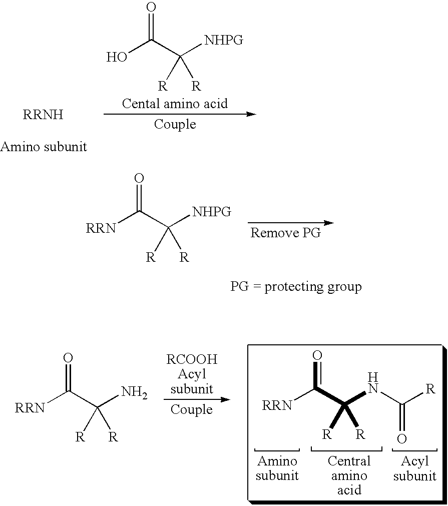

Various compounds of the invention may contain a guanidinoaryl moiety. This functionality may generally be prepared as described in U.S. Provisional Patent Application Nos. 60/230,565 and 60/245,579 and in U.S. patent application Ser. No. 09/945,384, which are herein incorporated by reference.

The amino subunit starting materials may also include guanidinoarylamines or aryl amines containing a handle for introducing guanidino substituents after amide bond coupling. For example, azidoarylamines (J=N₃ in scheme below) may be used as the coupling precursors. After the remaining subunits are assembled, the azide may be sequentially treated with a reducing agent, an isocyanate, and an amine to give the desired guanidino substituent. Alternatively a monoprotected aryl diamine (J=NHPG′) may also be used. The guanidino unit may be attached by reacting the deprotected aryl amino moiety sequentially with thiophosgene, a first amine, and finally a second amine. The above-described process can be summarized in the following synthesis scheme:

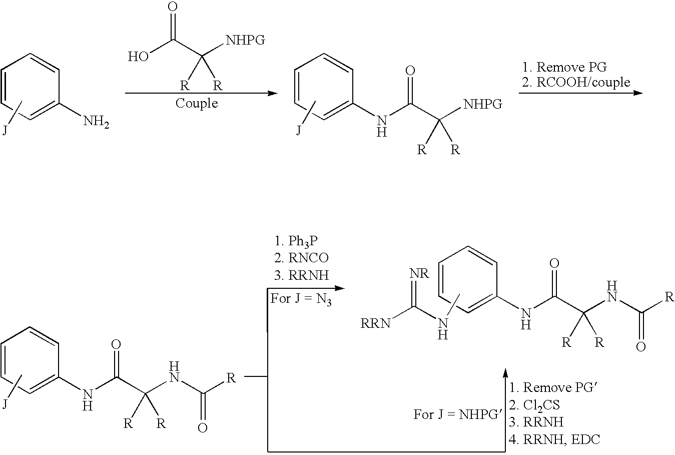

The starting materials for the central amino acid moiety may also be obtained from commercial sources such as Bachem (King of Prussia, Pa.) or may be prepared following know methods for the synthesis of unnatural amino acids. Representative examples of these methods may be found in the following reviews and articles: “Highly Practical Methodology for the synthesis of D- and L-α-Amino Acids, N-Protected α-Amino Acids, and N-Methyl-α-amino acids” (Myers, A. G.; Gleason, J. L.; Yoon, T.; Kung, D. W. J. Am. Chem. Soc, 1997, 119, 656-673), “Recent Developments in the Stereoselective Synthesis of α-aminoacids” (R. Duthaler, Tetrahedron, 1994, 50, p.1539-1650), and “Organic Chemistry Series Volume 7: Synthesis of Optically Active a-Amino Acids (Williams, R. M.; Permagon Press: Oxford, 1989).

The acyl subunit may also include a guanidinoaryl moiety. This functionality may be introduced by using azido-substituted or monoamine protected benzoic acids as the starting materials in the appropriate coupling step (see scheme below). The azide or protected amine may then be converted to a guanidino group in the same manner as described above.

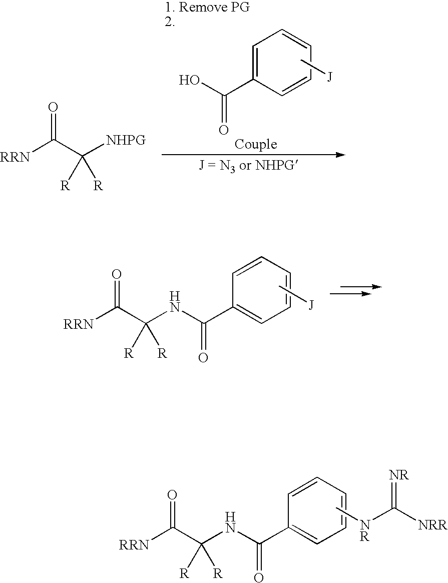

Other examples of starting materials for the acyl subunits include the bicyclic acids below prepared and described in WO 00/74679 and WO 99/64002.

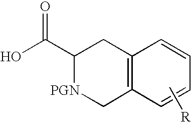

In one aspect of the instant invention the R substituent is a guanidino group or a latent guanidino group masked as a protected amine or as an azide. This temporary functionality may later be converted to a guanidino group in the same manner as described above. An example is shown in the scheme below.

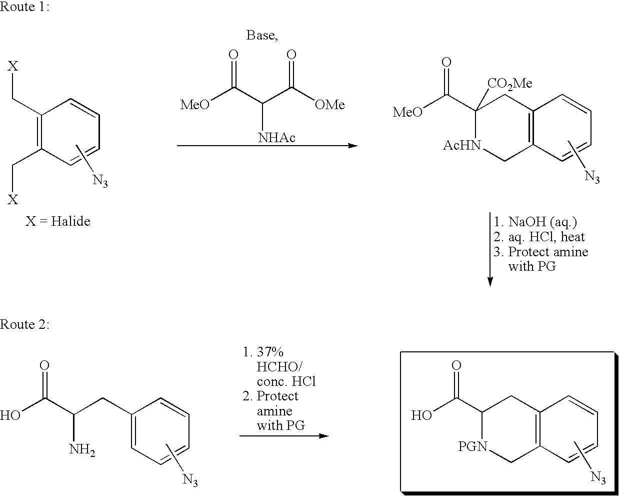

The instant invention also provides for compositions which may be prepared by mixing one or more compounds of the instant invention, or pharmaceutically acceptable salts or tautomers thereof, with pharmaceutically acceptable carriers, excipients, binders, diluents or the like, to treat or ameliorate a variety of disorders. Examples of such disorders include, but are not limited to obesity, erectile disorders, cardiovascular disorders, neuronal injuries or disorders, inflammation, fever, cognitive disorders, sexual behavior disorders. A therapeutically effective dose further refers to that amount of one or more compounds of the instant invention sufficient to result in amelioration of symptoms of the disorder. The pharmaceutical compositions of the instant invention can be manufactured by methods well known in the art such as conventional granulating, mixing, dissolving, encapsulating, lyophilizing, emulsifying or levigating processes, among others. The compositions can be in the form of, for example, granules, powders, tablets, capsules, syrup, suppositories, injections, emulsions, elixirs, suspensions or solutions. The instant compositions can be formulated for various routes of administration, for example, by oral administration, by intranasal administration, by transmucosal administration, by rectal administration, or subcutaneous administration as well as intrathecal, intravenous, intramuscular, intraperitoneal, intranasal, intraocular or intraventricular injection. The compound or compounds of the instant invention can also be administered in a local rather than a systemic fashion, such as injection as a sustained release formulation. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive or excipient such as a starch or other additive. Suitable additives or excipients are sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, a thickeners, buffers, a sweeteners, flavoring agents or perfuming agents. Additionally, dyestuffs or pigments may be added for identification. Tablets and pills may be further treated with suitable coating materials known in the art.

Liquid dosage forms for oral administration may be in the form of pharmaceutically acceptable emulsions, syrups, elixirs, suspensions, slurries and solutions, which may contain an inactive diluent, such as water. Pharmaceutical formulations may be prepared as liquid suspensions or solutions using a sterile liquid, such as, but not limited to, an oil, water, an alcohol, and combinations of these. Pharmaceutically suitable-surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For intranasal administration (e.g., to deliver compounds to the brain), or administration by inhalation (e.g., to deliver compounds through the lungs), the pharmaceutical formulations may be a solution, a spray, a dry powder, or aerosol containing any appropriate solvents and optionally other compounds such as, but not limited to, stabilizers, antimicrobial agents, antioxidants, pH modifiers, surfactants, bioavailability modifiers and combinations of these. Examples of intranasal formulations and methods of administration can be found in WO 01/41782, WO 00/33813, WO 91/97947, U.S. Pat. No. 6,180,603, and U.S. Pat. No. 5,624,898. A propellant for an aerosol formulation may include compressed air, nitrogen, carbon dioxide, or a hydrocarbon based low boiling solvent. The compound or compounds of the instant invention are conveniently delivered in the form of an aerosol spray presentation from a nebulizer or the like.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers.

For rectal administration, the pharmaceutical formulations may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed for to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations may be compressed into pellets or cylinders and implanted intramuscularly or subcutaneously as depot injections or as implants such as stents. Such implants may employ known inert materials such as silicones and biodegradable polymers.

A therapeutically effective dose refers to that amount of the compound that results in amelioration of symptoms. Specific dosages may be adjusted depending on conditions of disease, the age, body weight, general health conditions, sex, diet of the subject, dose intervals, administration routes, excretion rate, and combinations of drugs. Any of the above dosage forms containing effective amounts are well within the bounds of routine experimentation and therefore, well within the scope of the instant invention. A therapeutically effective dose may vary depending upon the route of administration and dosage form. The preferred compound or compounds of the instant invention is a formulation that exhibits a high therapeutic index. The therapeutic index is the dose ratio between toxic and therapeutic effects which can be expressed as the ratio between LD50 and ED50. The LD50 is the dose lethal to 50% of the population and the ED50 is the dose therapeutically effective in 50% of the population. The LD50 and ED50 are determined by standard pharmaceutical procedures in animal cell cultures or experimental animals.

The present invention also provides methods of enhancing MC4-R activity in a human or non-human animal. The method comprises administering an effective amount of a compound, or composition, of the instant invention to said mammal or non-human animal. Effective amounts of the compounds of the instant invention include those amounts that activate MC4-R which are detectable, for example, by an assay described below, or any other assay known by those skilled in the art that detect signal transduction, in a biochemical pathway, through activation of G-protein coupled receptors, for example, by measuring an elevated cAMP level as compared to a control model. Accordingly, “activating” means the ability of a compound to initiate a detectable signal. Effective amounts may also include those amounts which alleviate symptoms of a MC4-R disorder treatable by activating MC4-R.

An MC4-R disorder, or MC4-R-mediated disease, which may be treated by those methods provided, include any biological disorder or disease in which MC4-R is implicated, or which inhibition of MC4-R potentiates a biochemical pathway that is defective in the disorder or disease state. Examples of such diseases are obesity, erectile disorders, cardiovascular disorders, neuronal injuries or disorders, inflammation, fever, cognitive disorders, type II diabetes, polycystic ovary disease, Syndrome X, complications from obesity and diabetes, and sexual behavior disorders. In a preferred embodiment, the instant invention provides compounds, compositions, and methods effective for reducing energy intake and body weight; reducing serum insulin and glucose levels; alleviating insulin resistance; and reducing serum levels of free fatty acids. Accordingly, the instant invention is particularly effective in treating those disorders or diseases associated with obesity or type II diabetes.

“Treating” within the context of the instant invention, therefore, means an alleviation of symptoms associated with a disorder or disease, or halt of further progression or worsening of those symptoms, or prevention or prophylaxis of the disease or disorder. For example, within the context of obesity, successful treatment may include an alleviation of symptoms or halting the progression of the disease, as measured by reduction in body weight, or a reduction in amount of food or energy intake. In this same vein, successful treatment of type I or type II diabetes may include an alleviation of symptoms or halting the progression of the disease, as measured by a decrease in serum glucose or insulin levels in, for example, hyperinsulinemic or hyperglycemic patients.

EC₅₀ values of test compounds may be determined by treating cells expressing MC4-R with test compound and lysing the cells and measuring intercellular cAMP concentration with an Amersham-Pharmacia RPA-559 cAMP Scintillation Proximity Assay (SPA) kit. The compounds of the invention having the various formula IA through IXA are tested and display −log EC₅₀ values above about 3.

In vivo studies are conducted to observe the effect of MCR-4 agonists on energy intake, body weight, hyperinsulinemia, and glucose levels. All studies are conducted with male 9-10 week old ob/ob mice which display early onset of obesity, insulin resistance and diabetes due to leptin deficiency. Mice are acclimated in the facility for 1 week before studies and are caged individually. Vehicle-treated (control) and drug treated mice studies are always run in parallel. In multi-day studies, mice (8-15 per group) are monitored for baseline body weight, fasting levels of glucose, insulin, blood lipids and energy expenditure and then injected twice daily (9 a.m. and 5 p.m.) with 3 mg/kg of a MC4-R agonist of the present invention for 4 weeks. Body weight as well as food and water intake are monitored daily. Animals are fasted overnight for measurements of fasting levels of glucose, insulin, and lipids once a week until the end of the study. Energy expenditure (resting metabolic rate, i.e., O₂ consumption and CO₂ production) are monitored in air tight chambers at the end of the study on fed animals. O₂ consumption and CO₂ production are measured using Oxymax systems (Columbus Instruments). Oral glucose tolerance test (OGTT—a routine test for diabetes and glucose intolerance) is performed on overnight fasted mice at the end of the study. Blood glucose and oral glucose tolerance are measured using a glucose monitor (Onetouch sold by Lifescan). Free fatty acids are measured using an non-esterified free fatty acids enzymatic assay (Waco Chemicals). Serum Insulin levels are measured by immunoassay (Alpco).

The effect of the compounds of the present invention on food intake is determined by measuring grams/mouse/day throughout a 4 week study. Food is monitored every morning. Cumulative food intake represents the total amount of grams the mice consume during the study. A significant reduction in food intake is demonstrated in those mice treated IP with the compounds of the present invention.

The effect of the compounds of the present invention on body weight is determined by measuring grams/mouse throughout a 4 week study. Mice are weighed every morning. A significant body weight reduction is demonstrated in those mice treated IP with the compounds of the present invention.

The effect of the compounds of the present invention on blood glucose levels is determined by measuring blood glucose levels as represented as mg of glucose/dL of blood. Mice are fasted overnight and glucose levels are measured the following morning. Vehicle treated mice show an increase in blood glucose consistent with the rapid progression of diabetes in this mouse strain whereas, diabetes is slowed down considerably in drug treated mice. A significant reduction in fasting glucose levels is demonstrated in those mice treated IP with the compounds of this invention.

The effect of the compounds of the present invention on glucose levels during oral glucose tolerance test (OGTT) is determined by measuring blood glucose in overnight fasted mice. Blood glucose is represented as mg of glucose/dL of blood. Glucose levels are measured the following morning. Orally administered glucose quickly elevates blood glucose, similar to a meal, and the response to this exogenous glucose gives a measure of how well the body regulated glucose homeostasis. Vehicle treated mice show an elevated response to glucose consistent with their diabetic state, whereas drug treated mice show a very much improved glucose disposal.

The effect of the compounds of the present invention on free fatty acid (FFA) levels is determined by measuring mmoles of FFA/L of serum. Mice are fasted overnight and free fatty acid levels are measured the following morning. Vehicle treated mice show elevated levels of FFA throughout the study consistent with their obese state, whereas the drug treated mice diabetes show a dramatic decrease.

The effect of the compounds of the present invention on serum insulin levels is determined by measuring serum insulin levels one hour after single IP dosing of I and 3 mg/kg in overnight fasted ob/ob mice. Serum insulin levels are represented as ng of insulin/mL of serum. Drug treated mice show a dose dependent decrease relative to vehicle.

It is understood that the invention is not limited to the embodiments specifically set forth herein for illustration, but embraces all such forms thereof as would be understood by one of skill in the art and come within the scope of the following claims.

1. A compound of formula IA 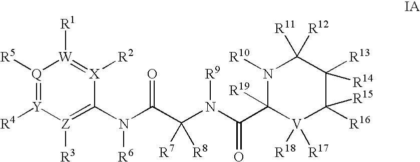
wherein
V is selected from a carbon atom or is absent from the ring such that the carbon atom bonded to R¹⁶ is bonded to the carbon atom bonded to R¹⁹ forming a 5-membered ring;
Q, W, X, Y, and Z are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 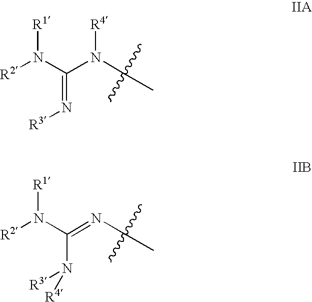
wherein R¹ may be absent if W is a nitrogen atom;
wherein R² may be absent if X is a nitrogen atom;
wherein R³ may be absent if Z is a nitrogen atom;
wherein R⁴ may be absent if Y is a nitrogen atom;
wherein R⁵ may be absent if Q is a nitrogen atom;
wherein one of R¹, R², R³, R⁴, or R⁵ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl,-alkynyl, cycloalkyl groups, heterocyclylalkyl groups, cycloalkylalkyl, aryl, heteroaryl groups, heterocyclyl groups, arylalkyl groups, and heteroarylalkyl groups;
R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups;
R⁹ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁰ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹¹, R¹², R¹³, R¹⁶, R¹⁷, and R¹⁸ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups; substituted and unsubstituted amide groups, substituted and unsubstituted sulfone groups, and substituted and unsubstituted sulfonamide groups;
wherein R¹³ and R¹⁶ together can represent a double bond between the carbon atoms bonded to R¹³ and R¹⁶;
wherein R¹⁷ and R¹⁸ are absent if V is absent;
R¹⁴ and R¹⁵ are selected from the group consisting of H, and substituted and unsubstituted alkyl groups;
or R¹⁴ and R¹⁵ together with the two carbon atoms to which they are bound form a substituted or unsubstituted, saturated or unsaturated, carbocyclic or heterocyclic ring comprising 5, 6, or 7 members;
R¹⁹ is selected from the group consisting of H, substituted and unsubstituted alkyl groups, substituted and unsubstituted cycloalkyl groups, and substituted and unsubstituted aryl groups; and
prodrugs thereof, pharmaceutically acceptable salts thereof, stereoisomers thereof, tautomers thereof, hydrates thereof, hydrides thereof, and solvates thereof.

2. A compound of claim 1, wherein one or more of R¹⁹, R⁶, R⁹, R¹⁰, R⁷, and R⁴ is H.

3. A compound of claim 1, wherein R⁸ is selected from the group consisting of substituted and unsubstituted arylalkyl groups, and substituted and unsubstituted heteroarylalkyl groups.

4. A compound of claim 1, wherein R^{3′} is selected from the group consisting of substituted and unsubstituted cycloalkyl, polycyclic cycloalkyl, alkenyl, alkyl, and aryl groups.

5. A compound of claim 1, wherein R^{1′} is H, and R^{2′} is selected from the group consisting of substituted and unsubstituted alkyl, arylalkyl, and heteroarylalkyl groups.

6. A compound of claim 1, wherein R^{1′} and R^{2′}, together with the nitrogen to which they are bound, form a substituted or unsubstituted heterocyclyl group.

7. A compound of claim 1, wherein R¹⁴ and R¹⁵ together with the two carbon atoms to which they are bound form a substituted or unsubstituted carbocyclic ring comprising 6 members.

8. A compound of claim 1, wherein Q is a carbon atom and R⁵ is a group of formula IIA or IIB.

9. A compound of formula IIIA: 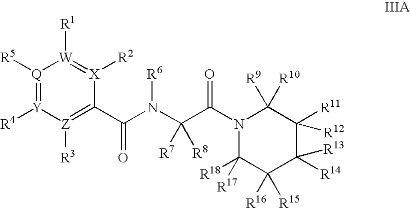
wherein
Q, W, X, Y, and Z are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 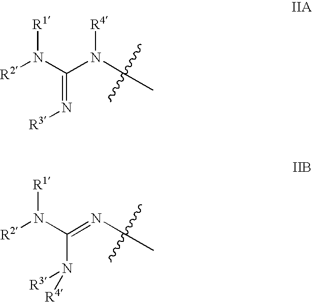
wherein R¹ may be absent if W is a nitrogen atom;
wherein R² may be absent if X is a nitrogen atom;
wherein R³ may be absent if Z is a nitrogen atom;
wherein R⁴ may be absent if Y is a nitrogen atom;
wherein R⁵ may be absent if Q is a nitrogen atom;
wherein one of R¹, R², R³, R⁴, or R⁵ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H and substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups;
R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups;
R⁹, R¹⁰, R¹¹, R¹², R¹⁵, R^{16, R}^{17, and R}¹⁸ are selected from the group consisting of H, Cl, F, Br, I, OH, —CN, —NO₂ substituted and unsubstituted alkoxy groups, and substituted and unsubstituted alkyl groups;
R¹³ and R¹⁴ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups; and substituted an unsubstituted alkylsulfonylalkyl groups;
or R¹³ and R¹⁴ may join together with the carbon atom to which they are bound to form a substituted or unsubstituted carbocyclic or heterocyclic ring; and
prodrugs thereof, pharmaceutically acceptable salts thereof, stereoisomers thereof, tautomers thereof, hydrates thereof, hydrides thereof, and solvates thereof.

10. A compound of claim 9, wherein one or more of R^{4′}, R⁶, and R⁷ is H.

11. A compound of claim 9, wherein R⁸ is selected from the group consisting of substituted and unsubstituted arylalkyl groups, and substituted and unsubstituted heteroarylalkyl groups.

12. A compound of claim 9, wherein R^{3′} is selected from the group consisting of substituted and unsubstituted cycloalkyl, polycyclic cycloalkyl, alkenyl, alkyl, and aryl groups.

13. A compound of claim 9, wherein R^{1′} is H and R^{2′} is selected from the group consisting of substituted and unsubstituted alkyl, arylalkyl, and heteroarylalkyl groups.

14. A compound of claim 9, wherein R^{1′} and R^{2′}, together with the nitrogen to which they are bound form, a substituted or unsubstituted heterocyclyl group.

15. A compound of claim 9, wherein R¹³ and R¹⁴, together with the carbon atom to which they are bound, form a substituted or unsubstituted carbocyclic or heterocyclic ring.

16. A compound of claim 9, wherein Q is a carbon atom and R⁵ is a group of formula IIA or IIB.

17. A compound of claim 9 having formula IIIB: 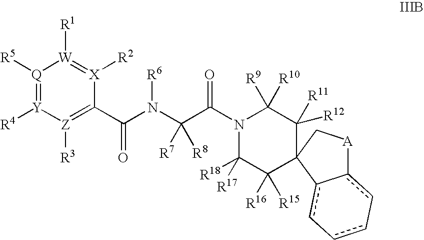
wherein the dotted lines in the 6-membered carbocyclic ring indicate that the 6-membered ring is a substituted or unsubstituted cyclohexyl or benzene ring; and
further wherein A is independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups.

18. A compound of formula IVA: 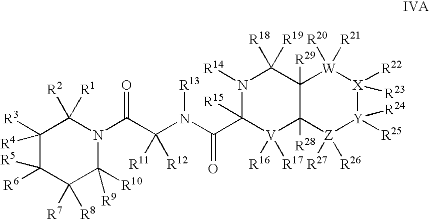
wherein
V is selected from a carbon atom or is absent from the ring such that the carbon atom bonded to R²⁸ is bonded to the carbon atom bonded to R¹⁵ forming a 5-membered ring.
W, X, Y, and Z are independently selected from the group consisting of carbon atoms and nitrogen atoms;
R¹, R², R³, R⁴, R⁷, R⁸, R⁹, and R¹⁰ are independently selected from the group consisting of H, Cl, F, Br, I, OH, —CN, —NO₂ substituted and unsubstituted alkoxy groups, and substituted and unsubstituted alkyl groups;
R⁵ and R⁶ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups; and substituted and unsubstituted alkylsulfonylalkyl groups;
wherein or R⁵ and R⁶ may join together with the carbon to which they are bound form a substituted or unsubstituted carbocyclic or heterocyclic ring;
R¹¹ is selected from the group consisting of H, substituted and unsubstituted alkyl groups, substituted and unsubstituted cycloalkyl groups, and substituted and unsubstituted aryl groups;
R¹² is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups;
R¹³ and R¹⁴ are independently selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁵, R¹⁶, R¹⁷, R¹⁸, and R¹⁹ are independently selected from H, substituted and unsubstituted alkyl groups, substituted and unsubstituted cycloalkyl groups, and substituted and unsubstituted aryl groups;
wherein R¹⁶ and R¹⁷ are absent if V is absent;
R²⁰, R²², R²⁴, and, R²⁶ are independently selected from the group consisting of H, substituted and unsubstituted alkyl groups, and groups having the formula IIA or IIB; 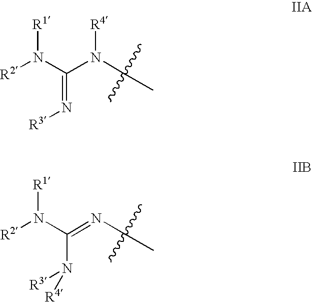
wherein R²⁰ may be absent if W is a nitrogen atom;
wherein R²² may be absent if X is a nitrogen atom;
wherein R²⁴ may be absent if Y is a nitrogen atom;
wherein R²⁶ may be absent if Z is a nitrogen atom;
wherein one of R²⁰, R²², R²⁴ or R²⁶ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups; and
R²¹, R²³, R²⁵, and R²⁷ are independently selected from the group consisting of H, substituted and unsubstituted alkyl groups, substituted and unsubstituted cycloalkyl groups, and substituted and unsubstituted aryl groups;
wherein R²¹ and R²³ together can represent a double bond between the carbons bonded to R²¹ and R²³;
wherein R²⁵ and R²⁷ together can represent a double bond between the carbons bonded to R²⁵ and R²⁷;
R²⁸ and R²⁹ are independently selected from the group consisting of H, and substituted and unsubstituted alkyl groups;
wherein R²⁸ and R²⁹ together can represent a double bond between the carbon atoms bonded to R²⁸ and R²⁹; and
prodrugs thereof, pharmaceutically acceptable salts thereof, stereoisomers thereof, tautomers thereof, hydrates thereof, hydrides thereof, and solvates thereof.

19. A compound of claim 18, wherein one or more of R^{4′}, R¹¹, R¹³, R¹⁴, and R¹⁵ is H.

20. A compound of claim 18, wherein R¹² is selected from the group consisting of substituted and unsubstituted arylalkyl groups, and substituted and unsubstituted heteroarylalkyl groups.

21. A compound of claim 18, wherein R^{3′} is selected from the group consisting of substituted and unsubstituted cycloalkyl, polycyclic cycloalkyl, alkenyl, alkyl, and aryl groups.

22. A compound of claim 18, wherein R^{1′} is H and R^{2′} is selected from the group consisting of substituted and unsubstituted alkyl, arylalkyl, and heteroarylalkyl groups.

23. A compound of claim 18, wherein R^{1′} is H and R^{2′} is selected from the group consisting of substituted and unsubstituted dialkylaminoethyl, 4-ethylbenzyl, 3-chlorobenzyl, 2,4-dichlorobenzyl, 3-methylbenzyl, benzyl, 4-fluorobenzyl, 3-methoxybenzyl, 2-chlorobenzyl, and thiophene groups.

24. A compound of claim 18, wherein R^{1′} and R^{2′}, together with the nitrogen to which they are bound, form a substituted or unsubstituted heterocyclyl group.

25. A compound of claim 18, wherein R⁵ and R⁶, together with carbon atom to which they are bound, form a substituted or unsubstituted carbocyclic or heterocyclic ring.

26. A compound of claim 18 having the formula IVB: 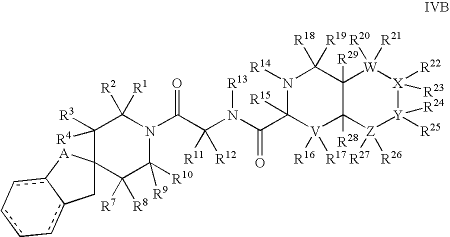
wherein the dotted lines in the 6-membered carbocyclic ring indicate that the 6-membered ring is a substituted or unsubstituted cyclohexyl or benzene ring; and
further wherein A is independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups.

27. A compound of formula VA: 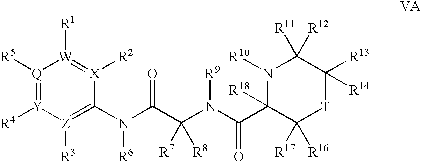
wherein
T is selected from the group consisting of O, S, and NR¹⁵ groups;
Q, W, X, Y, and Z are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 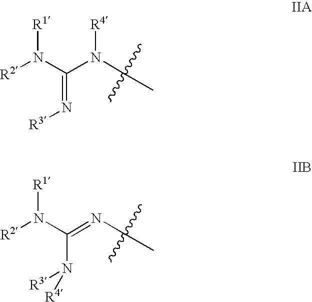
wherein R¹ may be absent if W is a nitrogen atom;
wherein R² may be absent if X is a nitrogen atom;
wherein R³ may be absent if Z is a nitrogen atom;
wherein R⁴ may be absent if Y is a nitrogen atom;
wherein R⁵ may be absent if Q is a nitrogen atom;
wherein one of R¹, R², R³, R⁴, or R⁵ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups;
R^{6′} is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups including arylalkyl groups substituted with groups of formula IIA or IIB, substituted and unsubstituted heteroarylalkyl groups including heteroarylalkyl groups substituted with groups of formula IIA or IIB, substituted and unsubstituted heterocyclylalkyl groups, substituted and unsubstituted cycloalkylalkyl groups, and substituted and unsubstituted aminoalkyl groups;
R⁹ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁰ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹¹, R¹², R¹³, R¹⁴, R¹⁶, and R¹⁷ are selected from the group consisting of H, Cl, F, Br, I, —CN, —OH, —NO₂, substituted and unsubstituted aryl groups, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups; substituted and unsubstituted amide groups, substituted and unsubstituted sulfone groups, and substituted and unsubstituted sulfonamide groups;
R¹⁵ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
or R¹² and R¹⁴ together with the carbon atoms to which they are bound, may form a substituted or unsubstituted, saturated or unsaturated carbocyclic or heterocyclic ring comprising 5 or 6 members;
or R¹⁴ and R¹⁵ together with the atoms to which they are bound, may form a substituted or unsubstituted, saturated or unsaturated heterocyclic ring comprising 5 or 6 members;
R¹⁸ is selected from the group consisting of H, substituted and unsubstituted alkyl groups, substituted and unsubstituted cycloalkyl groups, and substituted and unsubstituted aryl groups; and
prodrugs thereof, pharmaceutically acceptable salts thereof, stereoisomers thereof, tautomers thereof, hydrates thereof, hydrides thereof, and solvates thereof.

28. A compound of claim 27, wherein one or more of R¹⁸, R⁶, R⁹, R¹⁰, R⁷, and R^{4′} is H.

29. A compound of claim 27, wherein R⁸ is selected from the group consisting of substituted and unsubstituted arylalkyl groups, and substituted and unsubstituted heteroarylalkyl groups.

30. A compound of claim 27, wherein R^{3′} is selected from the group consisting of substituted and unsubstituted cycloalkyl, polycyclic cycloalkyl, alkenyl, alkyl, and aryl groups.

31. A compound of claim 27, wherein R^{1′} is H and R^{2′} is selected from the group consisting of substituted and unsubstituted alkyl, arylalkyl, and heteroarylalkyl groups.

32. A compound of claim 27, wherein R^{1′} and R^{2′}, together with the nitrogen to which they are bound, form a substituted or unsubstituted heterocyclyl group.

33. A compound of claim 27, wherein T is an NR¹⁵ group and R¹⁴ and R¹⁵ together with the atoms to which they are bound form a substituted or unsubstituted heterocyclic ring comprising 6 members.

34. A compound of claim 27, wherein Q is a carbon atom and R⁵ is a group of formula IIA or IIB.

35. A compound of formula VIA: 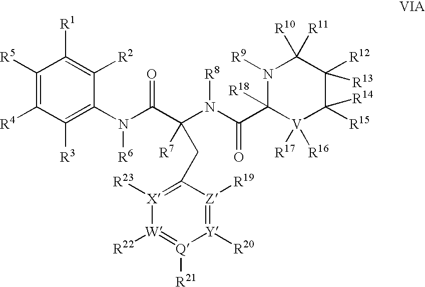
wherein
V is selected from a carbon atom or is absent from the ring such that the carbon atom bonded to R¹⁵ is bonded to the carbon atom bonded to R¹⁸ forming a 5-membered ring;
Q′, W′, X′, Y′, and Z′ are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB;
R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁹ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁰, R¹¹, R¹², R¹⁵, R¹⁶, and R¹⁷ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups; substituted and unsubstituted amide groups, substituted and unsubstituted sulfone groups, and substituted and unsubstituted sulfonamide groups;
wherein R¹² and R¹⁵ together can represent a double bond between the carbon atoms bonded to R¹² and R¹⁵;
wherein R¹⁶ and R¹⁷ are absent if V is absent;
R¹³ and R¹⁴ are selected from the group consisting of H, and substituted and unsubstituted alkyl groups;
or R¹³ and R¹⁴ together with the two carbon atoms to which they are bound form a substituted or unsubstituted, saturated or unsaturated, carbocyclic or heterocyclic ring comprising 5, 6, or 7 members;
R¹⁸ is selected from the group consisting of H, substituted and unsubstituted alkyl groups, substituted and unsubstituted cycloalkyl groups, and substituted and unsubstituted aryl groups;
R¹⁹, R²⁰, R²¹, R^{22,} and R²³ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 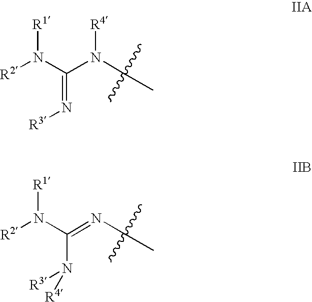
wherein R²² may be absent if W′ is a nitrogen atom;
wherein R²³ may be absent if X′ is a nitrogen atom;
wherein R¹⁹ may be absent if Z′ is a nitrogen atom;
wherein R²⁰ may be absent if Y′ is a nitrogen atom;
wherein R²¹ may be absent if Q′ is a nitrogen atom;
wherein one of R¹⁹, R²⁰, R²¹, R^{22,} or R²³ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;.
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups; and heteroarylalkyl group; and
prodrugs thereof, pharmaceutically acceptable salts thereof, stereoisomers thereof, tautomers thereof, hydrates thereof, hydrides thereof, and solvates thereof.

36. A compound of claim 35, wherein one or more of R^{4′}, R⁶, R⁷, R⁸, R⁹, and R¹⁸ is H.

37. A compound of claim 35, wherein R^{3′} is selected from the group consisting of substituted and unsubstituted cycloalkyl, polycyclic cycloalkyl, alkenyl, alkyl, and aryl groups.

38. A compound of claim 35, wherein R^{1′} is H and R^{2′} is selected from the group consisting of substituted and unsubstituted alkyl, arylalkyl, and heteroarylalkyl groups.

39. A compound of claim 35, wherein R^{1′} and R^{2′}, together with the nitrogen to which they are bound, form a substituted or unsubstituted heterocyclyl group.

40. A compound of claim 35, wherein R¹³ and R¹⁴ together with the two carbon atoms to which they are bound form a substituted or unsubstituted carbocyclic ring comprising 6 members.

41. A compound of claim 35, wherein Q′ is a carbon atom and R²¹ is a group of formula IIA or IIB.

42. A compound having the formula VIIA: 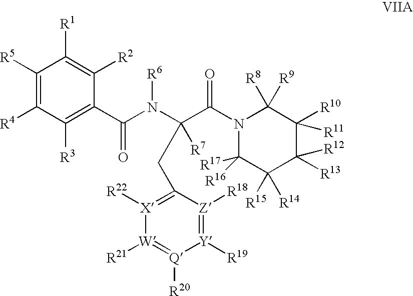
wherein
Q′, W′, X′, Y′, and Z′ are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB;
R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸, R⁹, R¹⁰, R¹, R¹⁴, R ⁵, R¹⁶, and R¹⁷ are selected from the group consisting of H, Cl, F, Br, I, OH, —CN, —NO₂ substituted and unsubstituted alkoxy groups, and substituted and unsubstituted alkyl groups;
R¹² and R¹³ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups; and substituted and unsubstituted alkylsulfonylalkyl groups;
wherein R¹² and R¹³ may join together with the carbon to which they are bound form a substituted or unsubstituted carbocyclic or heterocyclic ring including carbocyclic and heterocyclic rings substituted by a group of formula IIA or IIB;
R¹⁸, R¹⁹, R²⁰, R²¹, and R²² may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 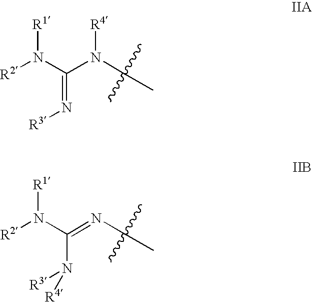
wherein R²¹ may be absent if W′ is a nitrogen atom;
wherein R²² may be absent if X′ is a nitrogen atom;
wherein R¹⁸ may be absent if Z′ is a nitrogen atom;
wherein R¹⁹ may be absent if Y′ is a nitrogen atom;
wherein R²⁰ may be absent if Q′ is a nitrogen atom;
wherein one of R¹⁸, R¹⁹, R²⁰, R²¹, or R²² is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H and substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups; and
prodrugs thereof, pharmaceutically acceptable salts thereof, stereoisomers thereof, tautomers thereof, hydrates thereof, hydrides thereof, and solvates thereof.

43. A compound of claim 42, wherein one or more of R^{4′}, R⁶, and R⁷ is H.

44. A compound of claim 42, wherein R^{3′} is selected from the group consisting of substituted and unsubstituted cycloalkyl, polycyclic cycloalkyl, alkenyl, alkyl, and aryl groups.

45. A compound of claim 42, wherein R^{1′} is H and R^{2′} is selected from the group consisting of substituted and unsubstituted alkyl, arylalkyl, and heteroarylalkyl groups.

46. A compound of claim 42, wherein R^{1′ and R}^{2′}, together with the nitrogen to which they are bound form a substituted or unsubstituted heterocyclyl group.

47. A compound of claim 42, wherein R¹² and R¹³, together with the carbon atom to which they are bound, form a substituted or unsubstituted carbocyclic or heterocyclic ring.

48. A compound of claim 42, wherein Q′ is a carbon atom and R²⁰ is a group of formula IIA or IIB.

49. A compound of claim 42 having formula VIIB: 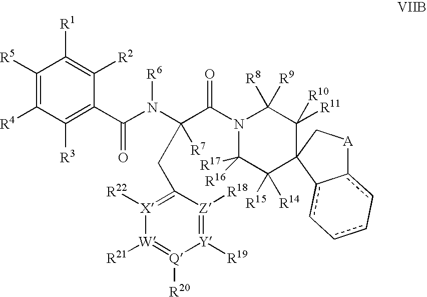
wherein the dotted lines in the 6-membered carbocyclic ring indicate that the 6-membered ring is a substituted or unsubstituted cyclohexyl or benezene ring; and
further wherein A is independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups.

50. A compound of formula VIIIA: 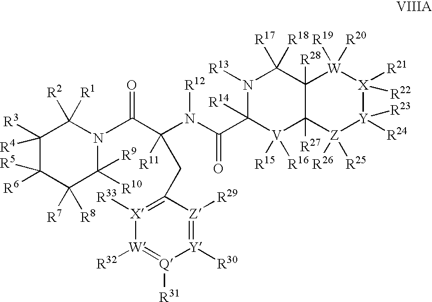
wherein
V is selected from a carbon atom or is absent from the ring such that the carbon atom bonded to R²⁷ is bonded to the carbon atom bonded to R¹ forming a 5-membered ring;
W, X, Y, and Z are independently selected from the group consisting of carbon atoms and nitrogen atoms;
Q′, W′, X′, Y′, and Z′ are independently selected from the group consisting of carbon atoms and nitrogen atoms;
R¹, R², R³, R⁴, R⁷, R⁸, R⁹, and R¹⁰ are independently selected from the group consisting of H, Cl, F, Br, I, OH, —CN, —NO₂ substituted and unsubstituted alkoxy groups, and substituted and unsubstituted alkyl groups;
R⁵ and R⁶ are independently selected from the group consisting of H, substituted and unsubstituted alkyl groups, substituted and unsubstituted cycloalkyl groups, substituted and unsubstituted aryl groups, substituted and unsubstituted arylalkyl groups, substituted and unsubstituted heterocyclylalkyl groups, substituted and unsubstituted —C(═O)—O(alkyl) groups, substituted and unsubstituted —C(═O)—O(aryl) groups, —C(═O)—OH groups, —C(═O)—NH₂ groups, substituted and unsubstituted —C(═O)—N(H)(alkyl) groups, substituted and unsubstituted —C(═O)—N(alkyl)₂ groups, substituted and unsubstituted —C(═O)—N(H)(aryl) groups, substituted and unsubstituted —C(═O)—N(aryl)(alkyl) groups, substituted and unsubstituted —C(═O)—N(aryl)₂ groups, substituted and unsubstituted —C(═O)—N(H)(heterocyclyl) groups, substituted and unsubstituted —C(═O)—N(alkyl)(heterocyclyl) groups, substituted and unsubstituted —C(═O)—N(aryl)(heterocyclyl) groups, substituted and unsubstituted —C(═O)—N(heterocyclyl)₂ groups; and substituted and unsubstituted alkylsulfonylalkyl groups;
wherein R⁵ and R⁶ may join together with the carbon to which they are bound form a substituted or unsubstituted carbocyclic or heterocyclic ring including carbocyclic or heterocyclic rings substituted with a group of formula IIA or IIB;
R¹¹ is selected from the group consisting of H, substituted and unsubstituted alkyl groups, substituted and unsubstituted cycloalkyl groups, and substituted and unsubstituted aryl groups;
R¹² and R¹³ are independently selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁴, R¹⁵, R¹⁶, R¹⁷, and R¹⁸ are independently selected from H, substituted and unsubstituted alkyl groups, substituted and unsubstituted cycloalkyl groups, and substituted and unsubstituted aryl groups;
wherein R¹⁵ and R¹⁶ are absent if V is absent;
R¹⁹, R²⁰, R²¹, R²², R²³, R²⁴, R²⁵, and R²⁶ are independently selected from the group consisting of H, substituted and unsubstituted alkyl groups, substituted and unsubstituted cycloalkyl groups, and substituted and unsubstituted aryl groups;
wherein R¹⁹ may be absent if W is a nitrogen atom;
wherein R²¹ may be absent if X is a nitrogen atom;
wherein R²³ may be absent if Y is a nitrogen atom;
wherein R²⁵ may be absent if Z is a nitrogen atom;
wherein R²⁰ and R²² together can represent a double bond between the carbons bonded to R²⁰ and R²²;
wherein R²⁴ and R²⁶ together can represent a double bond between the carbons bonded to R²⁴ and R²⁶;
R²⁷ and R²⁸ are independently selected from the group consisting of H, and substituted and unsubstituted alkyl groups;
wherein R²⁷ and R²⁸ together can represent a double bond between the carbon atoms bonded to R²⁷ and R²⁸;
R²⁹, R³⁰, R³¹, R³², and R³³ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups having the formula IIA or IIB; 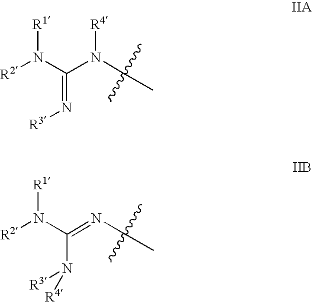
wherein R³¹ may be absent if Q′ is a nitrogen atom′
wherein R³² may be absent if W′ is a nitrogen atom;
wherein R³³ may be absent if X′ is a nitrogen atom;
wherein R³⁰ may be absent if Y′ is a nitrogen atom;
wherein R²⁹ may be absent if Z′ is a nitrogen atom;
wherein one of R²⁹, R³⁰, R³¹, R³², and R³³ is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups; and
prodrugs thereof, pharmaceutically acceptable salts thereof, stereoisomers thereof, tautomers thereof, hydrates thereof, hydrides thereof, and solvates thereof.

51. A compound of claim 50, wherein one or more of R⁴, R¹¹, R¹², and R¹³ is H.

52. A compound of claim 50, wherein R^{3′} is selected from the group consisting of substituted and unsubstituted cycloalkyl, polycyclic cycloalkyl, alkenyl, alkyl, and aryl groups.

53. A compound of claim 50, wherein R^{1′} is H and R^{2′} is selected from the group consisting of substituted and unsubstituted alkyl, arylalkyl, and heteroarylalkyl groups.

54. A compound of claim 50, wherein R^{1′} and R^{2′}, together with the nitrogen to which they are bound, form a substituted or unsubstituted heterocyclyl group.

55. A compound of claim 50, wherein R⁵ and R⁶ together with the carbon atom to which they are bound, form a substituted or unsubstituted carbocyclic or heterocyclic ring.

56. A compound of claim 50 having formula VIIIB: 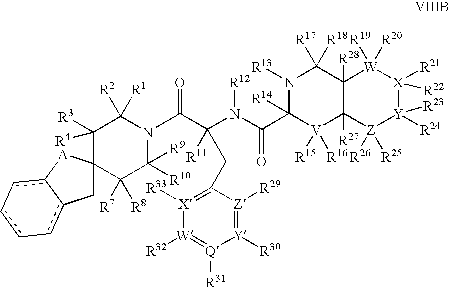
wherein the dotted lines in the 6-membered carbocyclic ring indicate that the 6-membered ring is a substituted or unsubstituted cyclohexyl or benzene ring; and
further wherein A is independently selected from the group consisting of a CH₂ group, a C(═O) group, a NH group, a substituted or unsubstituted N(alkyl) group, a C(H)(C(═O)—O(alkyl)) group, a C(H)(C(═O)—NH₂) group, a C(H)(C(═O)—N(H)(alkyl)) group, a C(H)(C(═O)—N(H)(aryl)) group, a C(H)(C(═O)—N(alkyl)₂) group, a C(H)(C(═O)—N(aryl)₂) group, substituted and unsubstituted alkoxyalkyl groups, and substituted and unsubstituted arylalkyl groups.

57. A compound of formula IXA: 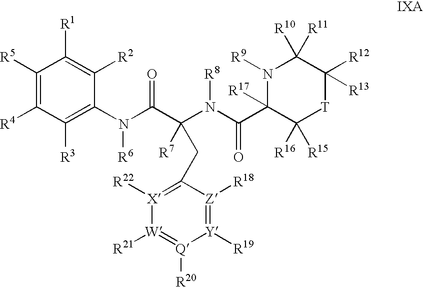
wherein
T is selected from the group consisting of O, S, and NR¹⁴ groups;
Q′, W′, X′, Y′, and Z′ are independently selected from the group consisting of carbon atoms and nitrogen atoms;
R¹, R², R³, R⁴, and R⁵ may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB;
R⁶ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted alkynyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁷ is selected from the group consisting of H, substituted and unsubstituted alkyl groups, substituted and unsubstituted cycloalkyl groups, and substituted and unsubstituted aryl groups;
R⁸ is selected from the group consisting H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R⁹ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
R¹⁰, R¹¹, R¹², R¹³, R¹⁵, and R¹⁶ are selected from the group consisting of H, Cl, F, Br, I, —CN, —OH, —NO₂, substituted and unsubstituted aryl, substituted and unsubstituted —C(═O)-alkyl groups, substituted and unsubstituted alkylcarbonylalkyl groups, substituted and unsubstituted alkoxy groups, substituted and unsubstituted aryloxy groups, substituted and unsubstituted alkyl groups, substituted and unsubstituted cycloalkyl groups, substituted and unsubstituted arylalkyl groups, substituted and unsubstituted heteroarylalkyl groups, substituted and unsubstituted heterocyclylalkyl groups, substituted and unsubstituted —NH₂ groups, substituted and unsubstituted N(H)(alkyl) groups, substituted and unsubstituted —NH(aryl) groups, substituted and unsubstituted —NH(heterocyclyl) groups, substituted and unsubstituted —N(alkyl)(aryl) groups, substituted and unsubstituted —N(alkyl)(heterocyclyl) groups, substituted and unsubstituted —N(aryl)(heterocyclyl) groups, substituted and unsubstituted —N(alkyl)₂ groups, substituted and unsubstituted —N(aryl)₂ groups, substituted and unsubstituted —N(heterocyclyl)₂ groups, —C(═O)—OH groups, substituted and unsubstituted —C(═O)—O(alkyl) groups; substituted and unsubstituted amide groups, substituted and unsubstituted sulfone groups, and substituted and unsubstituted sulfonamide groups;
R¹⁴ is selected from the group consisting of H, substituted and unsubstituted alkyl groups, substituted and unsubstituted cycloalkyl groups, substituted and unsubstituted aryl groups, substituted and unsubstituted heterocyclyl groups, substituted and unsubstituted heteroaryl groups, substituted and unsubstituted alkenyl groups, substituted and unsubstituted arylalkyl groups, substituted and unsubstituted heteroarylalkyl groups, substituted and unsubstituted heterocyclylalkyl groups, substituted and unsubstituted cycloalkylalkyl groups, and substituted and unsubstituted aminoalkyl groups;
or R¹¹ and R¹³ together with the carbon atoms to which they are bound, may form a substituted or unsubstituted, saturated or unsaturated carbocyclic or heterocyclic ring comprising 5 or 6 members;
or R¹³ and R¹⁴ together with the atoms to which they are bound, may form a substituted or unsubstituted, saturated or unsaturated heterocyclic ring comprising 5 or 6 members;
R¹⁷ is selected from the group consisting of H, substituted and unsubstituted alkyl groups, substituted and unsubstituted cycloalkyl groups, and substituted and unsubstituted aryl groups;
R¹⁸, R¹⁹, R²⁰, R²¹, and R²² may be the same or different, and are each independently selected from the group consisting of H, Cl, I, F, Br, OH, NH₂, CN, NO₂, and substituted and unsubstituted aryl, alkoxy, amino, alkyl, alkenyl, alkynyl, alkylamino, dialkylamino, cycloalkyl, heterocyclylamino, heteroarylamino, aminocarbonyl, alkylaminocarbonyl, dialkylaminocarbonyl, cycloalkylaminocarbonyl, arylaminocarbonyl, heterocyclylaminocarbonyl, heteroarylaminocarbonyl groups, and groups of formula IIA or IIB; 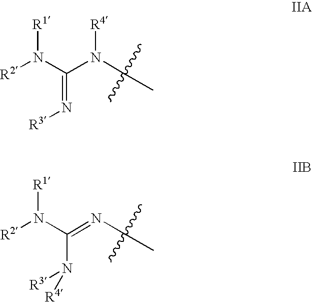
wherein R²¹ may be absent if W′ is a nitrogen atom;
wherein R²² may be absent if X′ is a nitrogen atom;
wherein R¹⁵ may be absent if Z′ is a nitrogen atom;
wherein R¹⁹ may be absent if Y′ is a nitrogen atom;
wherein R²⁰ may be absent if Q′ is a nitrogen atom;
wherein one of R¹⁸, R¹⁹, R²⁰, R²¹, and R²² is a group having the formula IIA or IIB;
R^{1′} is selected from the group consisting of H, and substituted and unsubstituted alkyl, alkenyl, alkynyl, cycloalkyl, aryl, heteroaryl, heterocyclyl, arylalkyl, heteroarylalkyl, and cycloalkylalkyl groups;
R^{2′} is selected from the group consisting of substituted and unsubstituted alkyl, alkenyl, alkynyl, cycloalkyl, aryl, heteroaryl, heterocyclyl, arylalkyl, heteroarylalkyl, and cycloalkylalkyl groups;
or R^{1′} and R^{2′}, together with the nitrogen to which they are bound, form a substituted or unsubstituted heterocyclyl or heteroaryl group;
R^{3′} is selected from the group consisting of H, substituted and unsubstituted aryl, alkyl, alkenyl, alkynyl, cycloalkyl, heteroaryl, heterocyclyl, heterocyclylalkyl, arylalkyl, heteroarylalkyl, and cycloalkylalkyl groups;
R^{4′} is selected from the group consisting of H, and substituted and unsubstituted alkyl, alkenyl, alkynyl, cycloalkyl groups, heterocyclylalkyl groups, cycloalkylalkyl, aryl, heteroaryl groups, heterocyclyl groups, arylalkyl groups, and heteroarylalkyl groups; and
prodrugs thereof, pharmaceutically acceptable salts thereof, stereoisomers thereof, tautomers thereof, hydrates thereof, hydrides thereof, and solvates thereof.

58. A compound of claim 57, wherein one or more of R^{4′}, R⁶, R⁷, R⁸, R⁹, and R¹⁷ is H.

59. A compound of claim 57, wherein R^{3′} is selected from the group consisting of substituted and unsubstituted cycloalkyl, polycyclic cycloalkyl, alkenyl, alkyl, and aryl groups.

60. A compound of claim 57, wherein R^{1′} is H and R^{2′} is selected from the group consisting of substituted and unsubstituted alkyl, arylalkyl, and heteroarylalkyl groups.

61. A compound of claim 57, wherein R^{1′} and R^{2′}, together with the nitrogen to which they are bound, form a substituted or unsubstituted heterocyclyl group.

62. A compound of claim 57, wherein T is an NR¹⁴ group and R¹³ and R¹⁴, together with the two atoms to which they are bound, form a substituted or unsubstituted heterocyclic ring comprising 6 members.

63. A compound of claim 57, wherein Q′ is a carbon atom and R²⁰ is a group of formula IIA or IIB.

64. A pharmaceutical formulation or medicament, comprising at least one of the compounds of any one of claims 1, 9, 18, 27, 35, 42, 50, or 57 and a pharmaceutically acceptable carrier.

65. A method of treating an MC4-R mediated disease, comprising administering to a subject in need thereof, at least one of the compounds of any one of claims 1, 9, 18, 27, 35, 42, 50, or 57.

66. A method of claim 65, wherein the MC4-R mediated disease is obesity or type II diabetes.
